SEQUENCE LISTING (58 sequences)

SEQ ID NO: 1 - DNA, 18 bp, human
cctggcacccagcacaat

SEQ ID NO: 2 - DNA, 20 bp, human
gccgatccacacggagtact

SEQ ID NO: 3 - DNA, 23 bp, human
tgactttgtcacagcccaagata

SEQ ID NO: 4 - DNA, 20 bp, human
aatgcggcatcttcaaacct

SEQ ID NO: 5 - DNA, 22 bp, human
aggatggcagctgagattctgt

SEQ ID NO: 6 - DNA, 24 bp, human
agagactgaagggtgtggaggtat

SEQ ID NO: 7 - DNA, 20 bp, human
cctgaacttgggtcccatca

SEQ ID NO: 8 - DNA, 19 bp, human
gccccaagctgctaaaagc

SEQ ID NO: 9 - DNA, 20 bp, human
tgcaaggctgacgataagga

SEQ ID NO: 10 - DNA, 26 bp, human
gtaggctgagatgcttttaaatgtga

SEQ ID NO: 11 - DNA, 25 bp, human
actccagagctgctaatctcattgt

SEQ ID NO: 12 - DNA, 26 bp, human
aactagtaagacaggtgggaggttct

SEQ ID NO: 13 - DNA, 25 bp, human
aaacaacttctggtggattcctgta

SEQ ID NO: 14 - DNA, 21 bp, human
gctctggatggtggatttcaa

SEQ ID NO: 15 - DNA, 25 bp, human
gctcttgaaaatggatacactttgc

SEQ ID NO: 16 - DNA, 27 bp, human
tctgagtttgaatctaggtctgcatag

SEQ ID NO: 17 - DNA, 21 bp, human
cccttctcctgttcctttgga

SEQ ID NO: 18 - DNA, 19 bp, human
cctttgcaggacgccttct

SEQ ID NO: 19 - DNA, 22 bp, human
ccagatccctgtgagcatgtac

SEQ ID NO: 20 - DNA, 22 bp, human
tgactgcgctgtctgatatcct

SEQ ID NO: 21 - DNA, 23 bp, human
catgctacgtgatgaagatggaa

SEQ ID NO: 22 - DNA, 23 bp, human
aacaaggaaaacattgccatctc

SEQ ID NO: 23 - DNA, 22 bp, human
aaagtgctggcccatttctatg

SEQ ID NO: 24 - DNA, 22 bp, human
tctccaagaacagcttgtgcat

SEQ ID NO: 25 - DNA, 22 bp, human
tggtttccaggagtgagattga

SEQ ID NO: 26 - DNA, 23 bp, human
tggaataaagagaggtggcaaaa

SEQ ID NO: 27 - DNA, 21 bp, human
tcagatgctgaggctcaagga

SEQ ID NO: 28 - DNA, 22 bp, human
tgtgtgacttccaggtcttgga

SEQ ID NO: 29 - DNA, 23 bp, human
ctgcagagagccattcactttct

SEQ ID NO: 30 - DNA, 24 bp, human
ggtgaaactgaagacaatgtgctt

SEQ ID NO: 31 - DNA, 20 bp, human
ggaccaacggctttcttcaa

SEQ ID NO: 32 - DNA, 22 bp, human
cataatgacgttccaggatcca

SEQ ID NO: 33 - DNA, 19 bp, human
gcttgggttcctggaacgt

SEQ ID NO: 34 - DNA, 19 bp, human
agccatgagggctcgtttc

SEQ ID NO: 35 - DNA, 21 bp, human
gcacagatggagaacgagcaa

SEQ ID NO: 36 - DNA, 19 bp, human
agcagggagcgaaggtgat

SEQ ID NO: 37 - DNA, 22 bp, human
gacactccccgaagtcttttgt

SEQ ID NO: 38 - DNA, 20 bp, human
tcatcaagactactgtggcc

SEQ ID NO: 39 - DNA, 22 bp, human
agccaatgatgagagcaatgag

SEQ ID NO: 40 - DNA, 21 bp, human
tggaattcacggctgactttg

SEQ ID NO: 41 - DNA, 20 bp, human
catgggtgtggtctcattgg

SEQ ID NO: 42 - DNA, 22 bp, human
caacactaggctgcaccactgt

SEQ ID NO: 43 - DNA, 25 bp, human
ctgctattctgaaattgcctacatg

SEQ ID NO: 44 - DNA, 30 bp, human
actgtaaactccgtaaaaactgcttattaa

SEQ ID NO: 45 - DNA, 23 bp, human
catgctacgtgatgaagatggaa

SEQ ID NO: 46 - DNA, 23 bp, human
aacaaggaaaacattgccatctc

SEQ ID NO: 47 - DNA, 20 bp, human
tgaagctgcagaaccaacga

SEQ ID NO: 48 - DNA, 21 bp, human
cgctctcccagtcatcacagt

SEQ ID NO: 49 - DNA, 20 bp, human
catcttagcggctgctgttg

SEQ ID NO: 50 - DNA, 18 bp, human
cttctttgcggccaccat

SEQ ID NO: 51 - DNA, 19 bp, human
acctgggaaggtggtgctt

SEQ ID NO: 52 - DNA, 22 bp, human
ttcttcacgatgacagctttgc

SEQ ID NO: 53 - DNA, 21 bp, human
cattgagccccggtatagaca

SEQ ID NO: 54 - DNA, 22 bp, human
tgaagaactcgctctggaacac

SEQ ID NO: 55 - DNA, 17 bp, human
cagccgggtgggtagga

SEQ ID NO: 56 - DNA, 21 bp, human
cgattacaacatgcggacaaa

SEQ ID NO: 57 - DNA, 24 bp, human
gcttccaagaaggagatcaaagac

SEQ ID NO: 58 - DNA, 19 bp, human
cgacgagggtcagctacca

(19) United States
(12) Patent Application Publication
(10) Pub. No.: US 2016/0222456 A1
(43) Pub. Date: Aug. 4, 2016

(54) URINE EXOSOME mRNAs AND METHODS OF USING SAME TO DETECT DIABETIC NEPHROPATHY

(71) Applicants: HITACHI CHEMICAL COMPANY LTD., Tokyo (JP); HITACHI CHEMICAL COMPANY AMERICA, LTD., Cupertino, CA (US); THE REGENTS OF THE UNIVERSITY OF CALIFORNIA, Oakland, CA (US)

(72) Inventors: Cindy YAMAMOTO, Irvine, CA (US); Melanie OAKES, San Juan Capistrano, CA (US); Masato MITSUHASHI, Irvine, CA (US); Kumar SHARMA, Del Mar, CA (US)

(21) Appl. No.: 15/135,353

(22) Filed: Apr. 21, 2016

Related U.S. Application Data
(63) Continuation-in-part of application No. 14/432,744, filed on Mar. 31, 2015.
(60) Provisional application No. 61/710,627, filed on Oct. 5, 2012.
(60) Provisional application No. 62/151,265, filed on Apr. 22, 2015.

(51) Int. Cl. C12Q 1/68

(52) U.S. Cl. CPC C12Q 1/6883; C12Q 2600/118; C12Q 2600/158

(57) ABSTRACT

Embodiments of the invention relate generally to methods of identifying subjects likely to develop diabetes-associated damage to the nephron, or subjects in the early stages of diabetic nephropathy. In particular, several embodiments relate to quantification of diabetic nephropathy-associated markers by isolating RNA isolated from vesicles from patient urine samples. The levels of the marker in a subject can be compared to levels in a population having normal nephron function and/or used to to track progression of diabetic nephropathy in said subject over time.

### RELATED CASES

The entire disclosure of each of the applications listed in the accompanying Application Data Sheet is incorporated by reference herein.

### REFERENCE TO SEQUENCE LISTING

The present application is being filed accompanied by a Sequence Listing in electronic format. The Sequence Listing is provided as a file entitled “ST25_Sequence_Listing_HITACHI_114P1”, was created on Apr. 21, 2016, and is 8.4 kilobytes in size. The information in the Sequence Listing is incorporated herein by reference in its entirety

### BACKGROUND

1. Field

The present disclosure relates generally to identification of various biomarkers associated with diabetic nephropathy, also known as diabetic kidney disease (DKD). Several embodiments relate to the diagnosis of diabetic nephropathy and more specifically, the present disclosure relates to the identification and use of mRNAs isolated from exosomes derived from urine and their use in the recognition and ongoing monitoring of diabetic nephropathy.

2. Description of Related Art

Broadly speaking, the kidney functions to filter the blood of various metabolic waste products and excess water. Every day, an adult's kidneys filter about 200 quarts of blood resulting in generation and excretion of about 2 quarts of waste products and extra water. The functional unit of the kidney is the nephron, and each kidney comprises about 1 million nephrons. Within each nephron is a glomerulus, which acts as the filtering mechanism, which keeps normal proteins and/or cells in the blood stream, and allows the excess water and waste to pass through to be processed by the remainder of the nephron (e.g., either concentrated or diluted). While some loss of kidney function can be tolerated—many individuals can lead normal lives with just one kidney—in many cases the cause of the reduction in kidney function is due to progressive disease or damage. In extreme cases, the progressive loss of kidney function results in the need for renal replacement therapy, such as dialysis or even kidney transplant. Two of the most common causes of loss of kidney function are diabetes and high blood pressure. According to recent data from the American Diabetes Association, over 8% of the United States population is diabetic. Over 200,000 people were living on chronic dialysis or with kidney transplant, due to end-stage kidney disease caused by diabetes. This comes at an extraordinary cost, not only to the patient's themselves, but to their family and the healthcare system.

### SUMMARY

Exosomes and microvesicles can be isolated from various biological fluids such as urine, blood and saliva. The RNA enclosed within these biological components is protected from degradation by nucleases and could be used as potential non-invasive sources of biomarkers. In several embodiments, the present disclosure relates to methods of collecting urine and other fluids and isolating exosomes and microvesicles in order to identify biomarkers with improved sensitivity for detecting diabetic kidney disease and other conditions.

In several embodiments, there is provided a method for identifying a subject likely to develop or currently affected by diabetic nephropathy, the method comprising obtaining a sample of urine from a subject, wherein the sample comprises vesicles that are associated with RNA, isolating the vesicles from the sample, lysing the vesicles to release the vesicle-associated RNA, wherein the vesicle-associated RNA comprises a target RNA, which is selected from the group consisting of PPARGC1A, SMAD1, UMOD, NRF2, SLC12A1, CD24, NFE2L2, FTH1, NDUFB2, RPL27, RPL30, ACTB, B2M, OAZ1, and RPS16, quantifying the target RNA, comparing the amount of the target RNA from the subject to the quantity of a corresponding RNA from individuals having normal kidney function, and identifying a subject as likely to develop or currently affected by diabetic nephropathy when there is a difference in the quantity of the target RNA between the subject and the quantity of the RNA in individuals with normal kidney function. In several embodiments the quantifying is performed by a method comprising contacting RNA from the sample with a reverse transcriptase to generate complementary DNA (cDNA) and (ii) contacting the cDNA with sense and antisense primers that are specific for one of PPARGC1A, SMAD1, UMOD, NRF2, SLC12A1, CD24, NFE2L2, FTH1, NDUFB2, RPL27, RPL30, ACTB, B2M, OAZ1, and RPS16 and a DNA polymerase to generate amplified DNA. Thereafter, the expression levels of the mRNA can be quantified (using the amplified DNA as a surrogate).

In several embodiments, the methods further comprise administering a therapy to subject, based on the outcome of the results. For example, a drug therapy may be administered, either alone or in conjunction with diet, exercise, or lifestyle changes. In addition, dialysis may also be administered. In some embodiments, a kidney transplant is performed.

There is also provided a method for identifying a subject affected by diabetic nephropathy, comprising obtaining a sample of urine from a subject, wherein the sample comprises vesicles that are associated with RNA, isolating the vesicles from the sample, lysing the vesicles to release the vesicle-associated RNA, wherein the vesicle-associated RNA comprises a target RNA, wherein the target RNA is selected from the group consisting of PPARGC1A, SMAD1, UMOD, NRF2, SLC12A1, CD24, NFE2L2, FTH1, NDUFB2, RPL27, RPL30, ACTB, B2M, OAZ1, and RPS16, quantifying the target RNA, and comparing the amount of the target RNA from the subject to the quantity of a corresponding RNA from individuals having normal kidney function, wherein a difference in the quantity of the target RNA between the subject and the individuals indicates the subject is affected by diabetic nephropathy.

Moreover, there is additionally provided a method for determining the progression of diabetic nephropathy in a patient comprising, obtaining a first sample of urine from a patient at a first time and a second sample of urine from the patient at a second time that is after the first time; wherein the samples comprise vesicles that are associated with RNA, isolating the vesicles from the samples, lysing the vesicles to release the vesicle-associated RNA, wherein the vesicle-associated RNA comprises at least one target RNA and at least one RNA that does not change in response to diabetes-induced damage to the nephron, wherein the at least one target RNA is selected from the group consisting of PPARGC1A, SMAD1, UMOD, NRF2, SLC12A1, CD24, NFE2L2, FTH1, NDUFB2, RPL27, RPL30, ACTB, B2M, OAZ1, RPS16, and combinations thereof, quantifying the at least one target RNA and the at least one RNA that does not change in response to diabetes-induced damage to the nephron, and determining a ratio between the amounts of the at least one target RNA and the at least one RNA that does not change in response to diabetes-induced damage to the nephron, and identifying progression in the patient's diabetic nephropathy when the ratio of the at least one RNA one target RNA to the at least one RNA that does not change in response to diabetes-induced damage to the nephron is increased in the second sample as compared to the first sample.

There is additionally provided, a nucleic-acid based method for detection of early stage diabetic nephropathy, comprising obtaining a sample of urine from a subject, wherein the sample comprises vesicles that are associated with RNA, isolating the vesicles from the sample, lysing the vesicles to release the vesicle-associated RNA, wherein the vesicle-associated RNA comprises a target RNA, wherein the target RNA is selected from the group consisting of PPARGC1A, SMAD1, UMOD, NRF2, SLC12A1, CD24, NFE2L2, FTH1, NDUFB2, RPL27, RPL30, ACTB, B2M, OAZ1, and RPS16, quantifying the target RNA, and comparing the amount of the target RNA from the subject to the quantity of a corresponding RNA from individuals having normal kidney function, wherein a difference in the quantity of the target RNA between the subject and the individuals indicates early stage diabetic nephropathy, thereby detecting early stage diabetic nephropathy. In several embodiments, the detection can be achieved prior to detection by non-nucleic acid detection methods.

In several embodiments, there is provided a method for advising a subject to undertake a therapy regime for diabetic nephropathy, comprising ordering a test of the subject's urine, the test comprising obtaining a sample of urine from a subject, wherein the sample comprises vesicles that are associated with RNA, isolating the vesicles from the sample, lysing the vesicles to release the vesicle-associated RNA, wherein the vesicle-associated RNA comprises a target RNA, wherein the target RNA is selected from the group consisting of PPARGC1A, SMAD1, UMOD, NRF2, SLC12A1, CD24, NFE2L2, FTH1, NDUFB2, RPL27, RPL30, ACTB, B2M, OAZ1, and RPS16, quantifying the target RNA, comparing the amount of the target RNA from the subject to the quantity of a corresponding RNA from individuals having normal kidney function, characterizing the subject as likely to develop or currently affected by diabetic nephropathy when there is a difference in the quantity of the target RNA between the subject and the quantity of the RNA in individuals with normal kidney function, and advising the subject to undertake a therapy for diabetic nephropathy when characterized as likely to develop or currently affected by diabetic nephropathy. In several embodiments, the therapy comprises one or more of diet, exercise, dialysis and/or lifestyle changes.

There is additionally provided a method for treating a subject having diabetic nephropathy comprising obtaining a sample of urine from a subject, wherein the sample comprises vesicles that are associated with RNA, isolating the vesicles from the sample, lysing the vesicles to release the vesicle-associated RNA, wherein the vesicle-associated RNA comprises a target RNA, wherein the target RNA is selected from the group consisting of PPARGC1A, SMAD1, UMOD, NRF2, SLC12A1, CD24, NFE2L2, FTH1, NDUFB2, RPL27, RPL30, ACTB, B2M, OAZ1, and RPS16, quantifying the target RNA, comparing the amount of the target RNA from the subject to the quantity of a corresponding RNA from individuals having normal kidney function, characterizing the subject as currently affected by diabetic nephropathy when there is a difference in the quantity of the target RNA between the subject and the quantity of the RNA in individuals with normal kidney function, and administering a therapy to the to treat the diabetic nephropathy. In several embodiments, the therapy comprises one or more of diet, exercise, dialysis and/or lifestyle changes.

In several embodiments, the quantifying of the RNA is achieved by using a method selected from the group consisting of reverse-transcription polymerase chain reaction (RT-PCR), real-time RT-PCR, RNA sequencing, northern blotting, fluorescence activated cell sorting, ELISA, and mass spectrometry. Other quantification methods may also optionally be used. In several embodiments, the quantifying comprises use of real-time RT-PCR.

In several embodiments, differences in the quantity of a target RNA are correlated with one or more non-molecular indicators of diabetic nephropathy. In such embodiments, an initial, molecular diagnosis can be corroborated through the use of non-molecular means.

In several embodiments, the target RNA is selected from the group consisting of PPARGC1A, SMAD1, UMOD, NRF2, SLC12A1, CD24, NFE2L2, FTH1, NDUFB2, RPL27, RPL30, ACTB, B2M, OAZ1, and RPS16. In several embodiments, these markers are not otherwise expressed or detectable until identified using the methods disclosed herein, thereby allowing early detection of damage to the nephron (e.g., prior to manifestation of established symptoms).

In several embodiments, the isolation of the vesicles from the sample comprises filtering the urine. In several embodiments, the filtration traps the vesicles on the filter. The lysing is performed while the vesicles are trapped on the filter.

In several embodiments, the methods further comprise centrifuging the urine sample (or samples) to remove cellular debris. In several embodiments, centrifugation is performed prior to isolating the vesicles. In several embodiments, the methods comprise concentrating the vesicles further by filtering the supernatant of the centrifuged urine.

In several embodiments, the diabetic nephropathy is due to Type I or Type II diabetes. In some embodiments, the Type I or Type II diabetes has not yet been diagnosed.

In several embodiments, the target RNA comprises poly(A)+RNA.

In several embodiments, the methods disclosed herein are used to screen a plurality of subjects to determine their likelihood of developing diabetic nephropathy and/or to detect early stage diabetic nephropathy. In several embodiments, the methods further comprise treating the subjects for prevent and/or treat diabetic nephropathy.

Additionally, there are provided, in several embodiments, kits for detection of diabetic nephropathy. For example, in several embodiments, there is provided a kit for detection of early stage diabetic nephropathy, comprising (a) a reverse transcriptase enzyme for generating complementary DNA (cDNA) from RNA isolated from a urine sample of a subject being evaluated for their diabetic nephropathy status, (b) at least one pair of sense and antisense primers specific for one of PPARGC1A, SMAD1, UMOD, NRF2, SLC12A1, CD24, NFE2L2, FTH1, NDUFB2, RPL27, RPL30, ACTB, B2M, OAZ1, and RPS16, and (c) a DNA polymerase for generating amplified DNA from the cDNA.

In several embodiments, the kit further comprises a filter for capturing vesicles from the urine sample. In several embodiments, the kit further comprises a lysis buffer for liberating RNA from the vesicles. In several embodiments, the kit further comprises an elution buffer for transporting RNA from the lysed vesicles to an analysis vessel. In several embodiments, the kit additionally comprises a microplate configured to receive the RNA. In some embodiments, the microplate comprises oligo(dT) in each well of the plate.

In several embodiments, the kit further comprises a DNA amplification buffer comprising Tris-HCl, magnesium chloride, potassium chloride, and adenine, thymine, guanine, and cytosine nucleotides.

In several embodiments, the kit further comprises at least one fluorescent probe complementary to a region within the amplified DNA of one of PPARGC1A, SMAD1, UMOD, NRF2, SLC12A1, CD24, NFE2L2, FTH1, NDUFB2, RPL27, RPL30, ACTB, B2M, OAZ1, and RPS16. In several such embodiments, the kit optionally comprises a means for detecting the fluorescent probe.

In several embodiments, the kits enable the detection of diabetes-induced damage to the nephron. In several embodiments, the diabetes-induced damage to the nephron is correlated with one or more non-molecular indicators of diabetic nephropathy.

In several embodiments, the kit further comprises control DNA indicating expression levels of one of PPARGC1A, SMAD1, UMOD, NRF2, SLC12A1, CD24, NFE2L2, FTH1, NDUFB2, RPL27, RPL30, ACTB, B2M, OAZ1, and RPS16 in the absence of diabetic nephropathy.

In several embodiments, the kits are suitable for detecting diabetic nephropathy that is due to Type I or Type II diabetes. In several embodiments, advantageously, the kits are able to detect diabetic nephropathy when associated Type I or Type II diabetes has not yet been diagnosed.

The methods summarized above and set forth in further detail below describe certain actions taken by a practitioner; however, it should be understood that they can also include the instruction of those actions by another party. Thus, actions such as “administering a blood test” include “instructing the administration of a blood test.”

### BRIEF DESCRIPTION OF THE FIGURES

FIGS. 1A-1H depict analysis of mRNA levels from control and diabetic patients.

FIGS. 2A-2H depict analysis of mRNA levels correlated with blood levels of HbA1c.

FIGS. 3A-3H depict analysis of mRNA levels correlated with serum creatinine levels.

FIGS. 4A-4C depict EMV sample prep for RNA sequencing.

FIG. 5 depicts an mRNA amplification scheme.

FIGS. 6A-B depict chromosomal gene alignment.

FIG. 7 depicts functional annotation of differentially expressed genes.

FIGS. 8A-8H depicts analysis of mRNA levels from control and DKD patients for the genes NFE2L2, FTH1, NDUFB2, RPL27, RPL30, ACTB, B2M, OAZ1, and RPS16.

### DETAILED DESCRIPTION

### General

Interpretation of a patient's symptoms, evaluation of their medical history and performance of a physical exam are typically used to generate an initial diagnosis, which is often corroborated with one or more medical tests. Many diagnostic medical tests are performed on blood extracted from a patient, as obtaining the sample is a relatively non-invasive procedure. Tests may measure concentrations of certain molecules in the sample, which are then compared to normal concentrations (e.g., healthy ranges) or to concentrations measured in a prior sample from the patient. Other tests may also be available to evaluate kidney function, such as measurement of glomerular filtration rate (GFR) or clearance of certain pharmacological marker compounds (e.g., analysis of urine samples over time). Another prognostic marker for kidney function is proteinuria, an elevated level of protein in the urine. Increasing amounts of proteins (such as albumin) in the urine indicate progressively increasing amounts of kidney damage, and associated loss of function. Unfortunately, many diagnostic tests are only sufficiently sensitive to detect measurable increases in a molecule (or molecules) associated with a disease or injury at such a time when significant disease progression or injury has already occurred. For example, in the context of evaluating kidney function, plasma concentrations of molecules such as creatinine or urea (waste substances that should be removed by a functional kidney) often will not be raised above the normal range until a substantial amount (e.g., 40% or greater) of total kidney function is lost.

Some of the assays described above rely on antigen-based detection of a marker or molecule, which can introduce some limitations with respect to sensitivity of the assay. Some assays employ more simplistic chemical reactions (e.g., colorimetric changes) to identify markers from blood or other fluid samples, however, these too may suffer from limitations of sensitivity. Questionable assay accuracy at low assay target concentration ranges significantly limits the ability of these assays to detect early stages of disease of injury that compromise kidney function. Thus, there exists a need for a sensitive, accurate and reproducible diagnostic test for evaluating kidney function that enable early detection and/or diagnosis of compromised kidney function.

### Vesicle-Associated RNA

As discussed in more detail below, several embodiments of the methods disclosed herein are based on the identification of specific markers that are indicative of disease or injury to the kidney, such as markers associated with diabetes-induced damage to the nephron. Although these markers can be identified through any of a number of different assays, the inventors have discovered that these markers can be determined sensitively, accurately and in a reproducible manner by using certain specific assay steps for evaluating the presence of RNA encoding these markers, which are isolated in a specific manner from vesicles. Advantageously, the nucleic acid-based methods provide a higher degree of sensitivity than the alternative assays disclosed above. In several embodiments, the nucleic acids are isolated from cells that are obtained from a blood or urine sample. In other embodiments, the nucleic acids exist extracellularly and are collected in a cell-free preparation. While several embodiments disclosed herein are directed to the isolation of RNA associated with vesicles present in patient urine samples, in several embodiments, RNA (and the associated markers) that are normally found in blood or plasma are isolated from urine samples. In some embodiments, these blood-borne markers are present in the urine due to damage or disease of the kidney that has compromised the normal blood filtering function of the kidney.

In several embodiments disclosed herein, there are provided methods for the capture of RNA from a sample of patient body fluid and subsequent analysis of that RNA for disease and/or tissue specific markers. In several embodiments, nucleic acids are associated with extracellular vesicles. In several embodiments, diagnosis and characterization of kidney disease/function is performed by detection, isolation, and quantification of specific RNA species from RNA-containing vesicles isolated from patient samples (e.g., urine). Exosomes and microvesicles are nm-sized particles that are shed from all cell types into biofluids such as plasma, cerebrospinal fluid, sputum, mucus, tears, saliva, ascites, and urine. They contain proteins and nucleic acids such as miRNA and mRNA which are representative of the cells in which they are derived from. For example, nucleic acids can be associated with one or more different types of membrane particles (ranging in size from 50-80 nm), exosomes (ranging in size from 50-100 nm), exosome-like vesicles (ranging in size from 20-50 nm), and microvesicles (ranging in size from 100-1000 nm). In several embodiments, these vesicles are isolated and/or concentrated, thereby preserving vesicle associated RNA even if there is a high RNAse extracellular environment. The RNAs within these particles have been shown to be functional and can confer specific activity to target cells. In several embodiments, the sensitivity of methods disclosed here is improved (vis-à-vis isolation of nucleic acids from tissues and/or collection of naked nucleic acids) based on the use of the vesicle-associated RNA. Many diagnostic tests are designed around using a small patient fluid sample, and in some embodiments, a small amount (e.g. 15-50 mL of urine) is used. However, several embodiments are particularly advantageous because large volumes of patient urine are readily available.

As described below, in some embodiments, the nucleic acids are vesicle-associated. In some embodiments, the nucleic acids detected are indicative of kidney disease and/or function (e.g., they not normally present in the urine of subject's having normal kidney function). In some embodiments, the detection of the nucleic acids is associated with severity and/or progression of kidney disease or injury (e.g., the nucleic acids are present in the patient urine sample at a greater or lesser concentration as compared to a population of individuals known to have normal kidney function). In some embodiments, urine is collected and nucleic acids are evaluated over time (e.g., to monitor a patient's response to therapy or disease progression).

RNA (and other nucleic acids) are typically within the intracellular environment. However, certain nucleic acids exist extracellularly. For example, in several embodiments, the methods involve collection and analysis of naked extracellular nucleic acids (e.g., naked RNA). This is advantageous in several embodiments because, typically, the extracellular environment that comprises substantial quantities of RNAses leads to rapid degradation of the nucleic acids.

A variety of methods can be used, according to the embodiments disclosed herein, to efficiently capture and preserve vesicle associated RNA. In several embodiments, centrifugation on a density gradient to fractionate the non-cellular portion of the sample is performed. In some embodiments, density centrifugation is optionally followed by high speed centrifugation to cause vesicle sedimentation or pelleting. As such approaches may be time consuming and may require expensive and specialized equipment in several embodiments, low speed centrifugation can be employed to collect vesicles.

In several embodiments, filtration (alone or in combination with centrifugation) is used to capture vesicles of different sizes. In some embodiments, differential capture of vesicles is made based on the surface expression of protein markers. For example, a filter may be designed to be reactive to a specific surface marker (e.g., filter coupled to an antibody) or specific types of vesicles or vesicles of different origin. In one embodiment, such vesicles are trapped on a filter, thereby allowing RNA extraction from the vesicles. In several embodiments, the combination of filtration and centrifugation allows a higher yield or improved purity of vesicles.

In some embodiments, the markers are unique vesicle proteins or peptides. In some embodiments, the severity of a particular disease or disorder is associated with certain vesicle modifications which can be exploited to allow isolation of particular vesicles. Modification may include, but are not limited to addition of lipids, carbohydrates, and other molecules such as acylated, formylated, lipoylated, myristolylated, palmitoylated, alkylated, methylated, isoprenylated, prenylated, amidated, glycosylated, hydroxylated, iodinated, adenylated, phosphorylated, sulfated, and selenoylated, ubiquitinated. In some embodiments, the vesicle markers comprise non-proteins such as lipids, carbohydrates, nucleic acids, RNA, DNA, etc.

In several embodiments, the specific capture of vesicles based on their surface markers also enables a “dip stick” format where each different type of vesicle is captured by dipping probes coated with different capture molecules (e.g., antibodies with different specificities) into a patient urine sample.

According to various embodiments, various methods to quantify RNA are used, including Northern blot analysis, RNAse protection assay, PCR, nucleic acid sequence-based amplification, branched-DNA amplification, ELISA, mass spectrometry, CHIP-sequencing, and DNA or RNA microarray analysis.

### Kidney Structure, Function, and Disease

The anatomy of the kidney is divided into two main tissue types, the renal cortex (the superficial area) and the renal medulla (the more interior area). Nephrons, the functional unit of the kidney, span the cortex and medulla. The initial filtering portion of a nephron is the renal corpuscle, located in the cortex, which leads to a renal tubule that passes from the cortex deep into the medulla.

A portion of the renal corpuscle, the glomerulus, performs the first step in filtering blood to form urine. The unique anatomy of the kidney leads to a high back-pressure in the glomerulus (due to the glomerulus draining into an arteriole rather than a venule). The back-pressure aids in the filtration process, but also has the potential to lead to kidney damage in certain disease states. Diabetes is characterized by elevated levels of glucose, a relatively large solute, in the blood. When diabetes is uncontrolled, the excess glucose can lead to physical damage to the glomerulus, which is exacerbated over time, as the initial damage to the glomerulus allows increased blood flow speed through the glomerulus, which results in the potential for further glucose-derived damage to the glomerulus.

After passing through the glomerulus, filtrate passes through the proximal tubule, the loop of Henle, the distal convoluted tubule, and the collecting duct. In sum, these anatomical structures function to generate a concentration gradient from the cortex to the medulla, which allows for the reabsorption of water from the filtrate, which creates concentrated urine for excretion.

Common clinical conditions involving the kidney include nephritic damage (either to the glomerulus specifically or the kidney generally), renal cysts, acute kidney injury, chronic kidney disease, urinary tract infection, nephrolithiasis (kidney stones), and urinary tract obstruction. Various cancers of the kidney exist, including, but not limited to, renal cell carcinoma, Wilms tumor, and renal cell carcinoma.

Several embodiments described herein are advantageous because markers associated with kidney function and/or disease can be rapidly assessed in a high through put protocol. Several embodiments are used to diagnose and/or monitor various kidney diseases (or loss of function related thereto), including, but not limited to chronic kidney disease, acute renal failure, diabetic nephropathy, glomerulonephritis, glomerulosclerosis, focal segmental glomerulosclerosis, membranous nephropathy, minimal change disease, and kidney disease secondary to other diseases such as atherosclerosis, hypertension, cardiovascular diseases, obesity, hypercholesterolemia, diabetes (e.g., diabetic nephropathy), collagen diseases, as well as kidney damage caused by pharmaceuticals or other compounds.

In several embodiments, damage to the kidney vasculature, in particular the endothelium of renal blood vessels, is detected by evaluation of kidney endothelial cell-specific mRNA. In some embodiments of the invention the markers are related to blood homeostasis such as endothelia cell marker von Willebrand factor (VWF), thrombin, factor VIII, plasmin, and fibrin. Von Willebrand factor is a plasma glycoprotein that is a mediator of platelet adhesion, as such it is released when the endothelium is damaged. VWF is involved in platelet aggregation and thrombus formation. In some embodiments, the markers may be kidney markers, such as, for example, Tamm-Horsfall glycoprotein (THP) also known as uromodulin, renin, solute carrier transporters (including, among others, SLC12A1, SLC22A6, SLC22A8, and SLC22A12), uromodulin associated kidney disease marker (UMOD), osteopontin (SPP1), and albumin (ALB), kidney fibrosis markers, such as matrix metallopeptidase 1 (MMP1) and matrix metallopeptidase 3 (MMP3), glomerular markers (e.g., glomerulus-specific (podocine (PDCN)), proximal tubule markers (e.g., proximal tubule-specific (uromodulin (UMOD)), albumin (ALB), Na/K/Cl transporter (SLC12A1)), distal tubule-specific markers (e.g., aquaporin 9 (AQP9)), as kidney-diabetes related markers including but not limited to peroxisome proliferator-activated receptor gamma, coactivator 1 α and β (PPARGC1A and B), nuclear respiratory factor 1 and 2 (NRF1 and 2), estrogen-related receptor α (ESRRA), annexin A5 (ANXA5), protein kinase, AMP-activated, α₁ and α□₂ catalytic subunit (PRKAA1 and 2), uncoupling protein 1 and 2 (UCP1 and 2), low density lipoprotein receptor-related protein 2 (LRP2), CD24, secreted phosphoprotein 1 (SPP1), α2-HS-glycoprotein (AHSG), SMAD family member 1 (SMAD1)) and the like. In some embodiments, the marker can be nuclear factor (erythroid-derived 2)-like 2 (NFE2L2), ferritin heavy peptide 1 (FTH1), ribosomal protein L30 (RPL30), ribosomal protein L27 (RPL27), NADH Dehydrogenase (ubiquinone) 1 Beta Subcomplex 2 (NDUFB2), ornithine decarboxylase antizyme 1 (OAZ1), ribosomal protein S16 (RPS16), and/or beta-2-microglobulin (B2M).

Several embodiments of the methods disclosed herein provide unexpected advantages over existing diagnostic and monitoring methods. For example, some diagnostic tests for kidney disease require a kidney biopsy, which is typically performed via puncture of the organ with a needle. The biopsy technique has the associated risks such as uncontrolled bleeding and infection. The methods described herein provide an opportunity to non-invasively identify RNA which indicates loss of kidney function due to diabetes (or other sources of kidney damage). Several embodiments thus unexpectedly enable remote sampling and assessment of the kidney without the associated increase in patient risk.

In addition to directly detecting direct kidney disease or injury, several embodiments of the methods disclosed herein are particularly advantageous because they are used to correlate a loss of kidney function (or symptoms thereof) with other diseases that are not kidney specific, but secondarily impact the kidney, for example, diabetes mellitus.

As discussed above, elevate blood glucose levels can lead to kidney damage and eventual reduction in kidney function. In some cases, diabetes mellitus leads to development of or is associated with one or more types of cardiovascular disease, which can further exacerbate kidney damage. In a healthy individual with normal functioning metabolism, insulin is produced by beta cells of the pancreas. The subsequent insulin release enables cells to absorb glucose. In contrast, in a diseased state the cells do not absorb glucose and it accumulates in the blood. This may lead to complications and/or damage to the kidney, as well as complications such as cardiovascular disease (coronary artery disease, peripheral vascular disease, and hypertension). Depending on the type of diabetes, a patient with diabetes either does not produce enough insulin or their cells do not properly respond to the insulin that their body does produce. In many cases, pre-diabetic individuals and/or those with diabetes live with early symptoms that are dismissed as being associated with other aspects of their lives or health. For example, post-prandial nausea may be ignored as heartburn, when in fact, the symptom is attributable to elevated blood glucose levels. Ignoring such symptoms over time can lead to, among other symptoms, excessive kidney damage prior to actual diagnosis. In several embodiments, the methods disclosed herein can be implemented in routine physical examinations to detect early markers of kidney damage due to diabetes before the symptoms become so severe that irreversible kidney damage is already sustained.

In several embodiments housekeeping gene products or constitutively expressed gene products, or markers of basal cellular function are used as markers or controls against which markers of diabetic nephropathy are compared. Housekeeping genes include, but are not limited to, glyceraldehyde 3-phosphate dehydrogenase, β actin (ACTB), and β2 microglobulin (B2M). Other housekeeping genes known in the art are used in other embodiments.

In several embodiments the functional status of a patient's kidneys is monitored over time, thereby allowing for the patient's kidney function be quantified at multiple time points. This data allows for tracking of the disease progress which in turn, in several embodiments, enables a medical professional to advise the patient with respect to what additional therapies and/or lifestyle changes might be required of a patient having a kidney disease, such as diabetic nephropathy.

In several such embodiments, a first sample of urine is collected from a patient and the level of vesicle or particle associated RNA for a specific gene or genes is determined. A subsequent sample (or samples) is collected from the patient and the level of specific RNA is determined. Any changes in kidney of the patient may thus be determined by comparing the first sample RNA level with the second sample RNA level or by comparing the samples to a control or standard. In some embodiments medication may have been administered to the patient before or after the collection of the first and/or second patient sample. In some embodiments, the medication may be a drug, nutritional supplement, vitamin, immunosuppressant, anti-inflammatory drug, anesthetic or analgesic, stem cell, graft, or kidney transplant. In some embodiments the monitoring may relate to a change in nutrition such as a reduction in caloric intake, or increased hydration, or change in exercise routine, or a change in sleeping pattern of the patient.

### Methodology

Free extracellular RNA is quickly degraded by nucleases, making it a potentially poor diagnostic marker. As described above, some extracellular RNA is associated with particles or vesicles that can be found in urine. This vesicle associated RNA, which includes mRNA, is protected from the degradation processes in the urine. Microvesicles are shed from most cell types and consist of fragments of plasma membrane. Microvesicles contain RNA, mRNA, microRNA, and proteins and mirror the composition of the cell from which they are shed. Exosomes are small microvesicles secreted by a wide range of mammalian cells and are secreted under normal and pathological conditions. These vesicles contain certain proteins and RNA including mRNA and microRNA. Exosomes can also be released into urine by the kidneys and their detection may serve as a diagnostic tool, as described in several embodiments herein. In addition to urine, exosome-like vesicles may also be found in many body fluids such as blood, ascites and amniotic fluid, among others. Several embodiments evaluate nucleic acids such as small interfering RNA (siRNA), tRNA, and small activating RNA (saRNA), among others.

In several embodiments the RNA isolated from vesicles from the urine of a patient with diabetic nephropathy is used as a template to make complementary DNA (cDNA). In several embodiments, cDNA is amplified using the polymerase chain reaction (PCR). In other embodiments, amplification of nucleic acid and RNA may also be achieved by any suitable amplification technique such as nucleic acid based amplification (NASBA) or primer-dependent continuous amplification of nucleic acid, or ligase chain reaction. Other methods may also be used to quantify the nucleic acids, such as for example, including Northern blot analysis, RNAse protection assay, PCR, nucleic acid sequence-based amplification, branched-DNA amplification, ELISA, mass spectrometry, CHIP-sequencing, and DNA or RNA microarray analysis.

In several embodiments, mRNA is quantified by a method entailing cDNA synthesis from mRNA and amplification of cDNA using PCR. In one preferred embodiment, a multi-well filterplate is washed with lysis buffer and wash buffer. A cDNA synthesis buffer is then added to the multi-well filterplate. The multi-well filterplate can be centrifuged. PCR primers are added to a PCR plate, and the cDNA is transferred from the multi-well filterplate to the PCR plate. The PCR plate is centrifuged, and real time PCR is commenced.

Another preferred embodiment comprises application of specific antisense primers during mRNA hybridization or during cDNA synthesis. It is preferable that the primers be added during mRNA hybridization, so that excess antisense primers may be removed before cDNA synthesis to avoid carryover effects. The oligo(dT) and the specific primer (NNNN) simultaneously prime cDNA synthesis at different locations on the poly-A RNA. The specific primer (NNNN) and oligo(dT) cause the formation of cDNA during amplification. Even when the specific primer-derived cDNA is removed from the GenePlate by heating each well, the amounts of specific cDNA obtained from the heat denaturing process (for example, using TaqMan quantitative PCR) is similar to the amount obtained from an un-heated negative control. This allows the heat denaturing process to be completely eliminated. Moreover, by adding multiple antisense primers for different targets, multiple genes can be amplified from the aliquot of cDNA, and oligo(dT)-derived cDNA in the GenePlate can be stored for future use.

Another alternative embodiment involves a device for high-throughput quantification of mRNA from biological fluid (e.g., urine). The device includes a multi-well filterplate containing: multiple sample-delivery wells, an exosome-capturing filter (or filter directed to another biological component of interest) underneath the sample-delivery wells, and an mRNA capture zone under the filter, which contains oligo(dT)-immobilized in the wells of the mRNA capture zone. In order to increase the efficiency of exosome collection, several filtration membranes can be layered together.

In some embodiments, amplification comprises conducting real-time quantitative PCR (TaqMan) with exosome-derived RNA and control RNA. In some embodiments, a TaqMan assay is employed. The 5′ to 3′ exonuclease activity of Taq polymerase is employed in a polymerase chain reaction product detection system to generate a specific detectable signal concomitantly with amplification. An oligonucleotide probe, nonextendable at the 3′ end, labeled at the 5′ end, and designed to hybridize within the target sequence, is introduced into the polymerase chain reaction assay. Annealing of the probe to one of the polymerase chain reaction product strands during the course of amplification generates a substrate suitable for exonuclease activity. During amplification, the 5′ to 3′ exonuclease activity of Taq polymerase degrades the probe into smaller fragments that can be differentiated from undegraded probe. In other embodiments, the method comprises: (a) providing to a PCR assay containing a sample, at least one labeled oligonucleotide containing a sequence complementary to a region of the target nucleic acid, wherein the labeled oligonucleotide anneals within the target nucleic acid sequence bounded by the oligonucleotide primers of step (b); (b) providing a set of oligonucleotide primers, wherein a first primer contains a sequence complementary to a region in one strand of the target nucleic acid sequence and primes the synthesis of a complementary DNA strand, and a second primer contains a sequence complementary to a region in a second strand of the target nucleic acid sequence and primes the synthesis of a complementary DNA strand; and wherein each oligonucleotide primer is selected to anneal to its complementary template upstream of any labeled oligonucleotide annealed to the same nucleic acid strand; (c) amplifying the target nucleic acid sequence employing a nucleic acid polymerase having 5′ to 3′ nuclease activity as a template dependent polymerizing agent under conditions which are permissive for PCR cycling steps of (i) annealing of primers and labeled oligonucleotide to a template nucleic acid sequence contained within the target region, and (ii) extending the primer, wherein said nucleic acid polymerase synthesizes a primer extension product while the 5′ to 3′ nuclease activity of the nucleic acid polymerase simultaneously releases labeled fragments from the annealed duplexes comprising labeled oligonucleotide and its complementary template nucleic acid sequences, thereby creating detectable labeled fragments; and (d) detecting and/or measuring the release of labeled fragments to determine the presence or absence of target sequence in the sample.

In alternative embodiments, a TaqMan assay is employed that provides a reaction that results in the cleavage of single-stranded oligonucleotide probes labeled with a light-emitting label wherein the reaction is carried out in the presence of a DNA binding compound that interacts with the label to modify the light emission of the label. The method utilizes the change in light emission of the labeled probe that results from degradation of the probe. The methods are applicable in general to assays that utilize a reaction that results in cleavage of oligonucleotide probes, and in particular, to homogeneous amplification/detection assays where hybridized probe is cleaved concomitant with primer extension. A homogeneous amplification/detection assay is provided which allows the simultaneous detection of the accumulation of amplified target and the sequence-specific detection of the target sequence.

In alternative embodiments, real-time PCR formats may also be employed. One format employs an intercalating dye, such as SYBR Green. This dye provides a strong fluorescent signal on binding double-stranded DNA; this signal enables quantification of the amplified DNA. Although this format does not permit sequence-specific monitoring of amplification, it enables direct quantization of amplified DNA without any labeled probes. Other such fluorescent dyes that may also be employed are SYBR Gold, YO-PRO dyes and Yo Yo dyes.

Another real-time PCR format that may be employed uses reporter probes that hybridize to amplicons to generate a fluorescent signal. The hybridization events either separate the reporter and quencher moieties on the probes or bring them into closer proximity. The probes themselves are not degraded and the reporter fluorescent signal itself is not accumulated in the reaction. The accumulation of products during PCR is monitored by an increase in reporter fluorescent signal when probes hybridize to amplicons. Formats in this category include molecular beacons, dual-hybe probes, Sunrise or Amplifluor, and Scorpion real-time PCR assays.

Another real-time PCR format that may also be employed is the so-called “Policeman” system. In this system, the primer comprises a fluorescent moiety, such as FAM, and a quencher moiety which is capable of quenching fluorescence of the fluorescent moiety, such as TAMRA, which is covalently bound to at least one nucleotide base at the 3′ end of the primer. At the 3′ end, the primer has at least one mismatched base and thus does not complement the nucleic acid sample at that base or bases. The template nucleic acid sequence is amplified by PCR with a polymerase having 3′-5′ exonuclease activity, such as the Pfu enzyme, to produce a PCR product. The mismatched base(s) bound to the quencher moiety are cleaved from the 3′ end of the PCR product by 3′-5′ exonuclease activity. The fluorescence that results when the mismatched base with the covalently bound quencher moiety is cleaved by the polymerase, thus removing the quenching effect on the fluorescent moiety, is detected and/or quantified at least one time point during PCR. Fluorescence above background indicates the presence of the synthesized nucleic acid sample.

Another alternative embodiment involves a fully automated system for performing high throughput quantification of mRNA in biological fluid (e.g., urine), including: robots to apply biological fluid samples, hypotonic buffer, and lysis buffer to the device; an automated vacuum aspirator and centrifuge, and automated PCR machinery.

The method of determining the presence of diabetic nephropathy may also employ other methods of measuring mRNA other than those described above. Other methods which may be employed include, for example, Northern blot analysis, Rnase protection, solution hybridization methods, semi-quantitative RT-PCR, and in situ hybridization.

In several embodiments, diabetic nephropathy induces the expression of one or more marker. In several embodiments, the increased expression is measured by the amount of mRNA encoding said markers (in other embodiments, DNA or protein are used to measure expression levels). In some embodiments urine is collected from a patient and directly evaluated. In some embodiments, vesicles are concentrated, for example by use of filtration or centrifugation. Isolated vesicles are then incubated with lysis buffer to release the RNA from the vesicles, the RNA then serving as a template for cDNA which is quantified with methods such as quantitative PCR (or other appropriate amplification or quantification technique). In several embodiments, the level of specific marker RNA from patient vesicles is compared with a desired control such as, for example, RNA levels from a healthy patient population, or the RNA level from an earlier time point from the same patient or a control gene from the same patient.

In several embodiments, the disclosed methods allow the detection of the presence or absence of diabetic nephropathy by measuring the levels of mRNA encoding one or more markers related to diabetic nephropathy. In several embodiments, the disclosed methods allow the assessment of the progression (or regression) of diabetic nephropathy by measuring the levels of mRNA encoding one or more markers related to diabetic nephropathy. To determine these mRNA levels, in some embodiments, mRNA-containing vesicles are isolated from plasma using a device for isolating and amplifying mRNA. Embodiments of this device are described in more detail in U.S. Pat. Nos. 7,745,180, 7,939,300, 7,968,288, 7,981,608, 8,076,105, 8,101,344, each of which is incorporated in its entirety by reference herein.

Certain embodiments comprise a multi-well plate that contains a plurality of sample-delivery wells, a vesicle-capturing filter underneath the wells, and an mRNA capture zone underneath the filter which contains immobilized oligo(dT). In certain embodiments, the device also contains a vacuum box adapted to receive the filter plate to create a seal between the plate and the box, such that when vacuum pressure is applied, the urine is drawn from the sample-delivery wells across the vesicle-capturing filter, thereby capturing the vesicles and allowing non-vesicle urine components to be removed by washing the filters. In other embodiments, other means of drawing the urine samples through the sample wells and through across the vesicle-capturing filter, such as centrifugation or positive pressure, are used. In some embodiments, vesicles are captured on a plurality of filter membranes that are layered together. In several embodiments, the captured vesicles are then lysed with a lysis buffer, thereby releasing mRNA from the captured vesicles. The mRNA is then hybridized to the oligo(dT)-immobilized in the mRNA capture zone. Further detail regarding the composition of lysis buffers that may be used in several embodiments can be found in U.S. Pat. No. 8,101,344, which is incorporated in its entirety by reference herein. In several embodiments, cDNA is synthesized from oligo(dT)-immobilized mRNA. In some embodiments, the cDNA is then amplified using real time PCR with primers specifically designed for amplification of disease-associated markers. Primers that are used in such embodiments are shown in Table 1. Further details about the PCR reactions used in some embodiments are also found in U.S. Pat. No. 8,101,344, which is incorporated in its entirety by reference herein.

TABLE 1
Primer Sequences for RT-PCR Amplification

| | FWD Sequence | REV Sequence |
|---|---|---|
| Target | (5′-3′) | (3′-5′) |
| β-Actin | CCTGGCACCCAGCACAAT | GCCGATCCACACGGAGTACT |
| | (SEQ ID No. 1) | (SEQ ID No. 2) |
| β-2 microglobulin | TGACTTTGTCACAGCCCAAGATA | AATGCGGCATCTTCAAACCT |
| (B2M) | (SEQ ID No. 3) | (SEQ ID No. 4) |
| PDCN (glomerulus | AGGATGGCAG CTGAGATTCT GT | AGAGACTGAA GGGTGTGGAG GTAT |
| specific podocin) | (SEQ ID No. 5) | (SEQ ID No. 6) |
| UMOD | CCTGAACTTG GGTCCCATCA | GCCCCAAGCT GCTAAAAGC |
| (uromodulin) | (SEQ ID No. 7) | (SEQ ID No. 8) |
| ALB | TGCAAGGCTGACGATAAGGA | GTAGGCTGAGATGCTTTTAAATGTGA |
| (albumin) | (SEQ ID No. 9) | (SEQ ID No. 10) |
| SLC12A1 | ACTCCAGAGCTGCTAATCTCATTGT | AACTAGTAAGACAGGTGGGAGGTTCT |
| (Na⁺/K⁺/C1⁻ | (SEQ ID No. 11) | (SEQ ID No. 12) |
| transporter) | | |
| AQP9 | AAACAACTTCTGGTGGATTCCTGTA | GCTCTGGATGGTGGATTTCAA |
| (distal tubule specific | (SEQ ID No. 13) | (SEQ ID No. 14) |
| aquaporin 9) | | |
| PPARGC1A | GCTCTTGAAAATGGATACACTTTGC | TCTGAGTTTGAATCTAGGTCTGCATAG |
| (peroxisome | (SEQ ID No. 15) | (SEQ ID No. 16) |
| proliferator-activated | | |
| receptor gamma, | | |
| coactivator 1 α) | | |
| PPARGC1B | CCCTTCTCCTGTTCCTTTGGA | CCTTTGCAGGACGCCTTCT |
| (peroxisome | (SEQ ID No. 17) | (SEQ ID No. 18) |
| proliferator-activated | | |
| receptor gamma, | | |
| coactivator 1 β) | | |
| NRF1 | CCAGATCCCTGTGAGCATGTAC | TGACTGCGCTGTCTGATATCCT |
| (nuclear respiratory | (SEQ ID No. 19) | (SEQ ID No. 20) |
| factor 1) | | |
| NRF2 | CATGCTACGTGATGAAGATGGAA | AACAAGGAAAACATTGCCATCTC |
| (nuclear respiratory | (SEQ ID No. 21) | (SEQ ID No. 22) |
| factor 2) | | |
| ESRRA | AAAGTGCTGGCCCATTTCTATG | TCTCCAAGAACAGCTTGTGCAT |
| (estrogen-related | (SEQ ID No. 23) | (SEQ ID No. 24) |
| receptor α) | | |
| ANXA5 | TGGTTTCCAGGAGTGAGATTGA | TGGAATAAAGAGAGGTGGCAAAA |
| (annexin 5) | (SEQ ID No. 25) | (SEQ ID No. 26) |
| PRKAA1 | TCAGATGCTGAGGCTCAAGGA | TGTGTGACTTCCAGGTCTTGGA |
| (AMP-activated, α1 | (SEQ ID No. 27) | (SEQ ID No. 28) |
| catalytic subunit) | | |
| PRKAA2 | CTGCAGAGAGCCA TTCACTTTCT | GGTGAAACTGAAGACAATGTGCTT |
| (AMP-activated, α2 | (SEQ ID No. 29) | (SEQ ID No. 30) |
| catalytic subunit) | | |
| UCP1 | GGACCAACGGCTTTCTTCAA | CATAATGACGTTCCAGGATCCA |
| (uncoupling protein | (SEQ ID No. 31) | (SEQ ID No. 32) |
| 1) | | |
| UCP2 | GCTTGGGTTCCTGGAACGT | AGCCATGAGGGCTCGTTTC |
| (uncoupling protein | (SEQ ID No. 33) | (SEQ ID No. 34) |
| 2) | | |
| LRP2 | GCACAGATGG AGAACGAGCA A | AGCAGGGAGC GAAGGTGAT |
| (low density | (SEQ ID No. 35) | (SEQ ID No. 36) |
| lipoprotein receptor- | | |
| related protein 2) | | |
| CD24 | GACACTCCCC GAAGTCTTTT GT | TCATCAAGAC TACTGTGGCC |
| | (SEQ ID No. 37) | ATATTAG |
| | | (SEQ ID No. 38) |
| SPP1 | AGCCAATGAT GAGAGCAATG AG | TGGAATTCAC GGCTGACTTT G |
| (secreted | (SEQ ID No. 39) | (SEQ ID No. 40) |
| phosphoprotein 1) | | |
| AHSG | CATGGGTGTGGTCTCATTGG | CAACACTAGGCTGCACCACTGT |
| (α2-HS-glycoprotein) | (SEQ ID No. 41) | (SEQ ID No. 42) |
| SMAD1 | CTGCTATTCT GAAATTGCCT | ACTGTAAACT CCGTAAAAAC |
| (SMAD family | ACATG | TGCTTATTAA |
| member 1) | (SEQ ID No. 43) | (SEQ ID No. 44) |
| NFE2L2 | CATGCTACGTGATGAAGATGGAA | AACAAGGAAAACATTGCCATCTC |
| (nuclear factor | (SEQ ID No. 45) | (SEQ ID No. 46) |
| (erythroid-derived-2)- | | |
| like 2) | | |
| FTH1 | TGAAGCTGCAGAACCAACGA | CGCTCTCCCAGTCATCACAGT |
| (ferritin, heavy | (SEQ ID No. 47) | (SEQ ID No. 48) |
| peptide 1) | | |
| RPL30 | CATCTTAGCGGCTGCTGTTG | CTTCTTTGCGGCCACCAT |
| (ribosomal protein | (SEQ ID No. 49) | (SEQ ID No. 50) |
| L30) | | |
| RPL27 | ACCTGGGAAGGTGGTGCTT | TTCTTCACGATGACAGCTTTGC |
| (ribosomal protein | (SEQ ID No. 51) | (SEQ ID No. 52) |
| L27) | | |
| NDUFB2 | CATTGAGCCCCGGTATAGACA | TGAAGAACTCGCTCTGGAACAC |
| (NADH | (SEQ ID No. 53) | (SEQ ID No. 54) |
| dehydrogenase | | |
| (ubiquinone) 1 beta | | |
| subcomplex 2) | | |
| OAZ1 | CAGCCGGGTGGGTAGGA | CGATTACAACATGCGGACAAA |
| (ornithine | (SEQ ID No. 55) | (SEQ ID No. 56) |
| decarboxylase | | |
| antizyme 1) | | |
| RPS16 | GCTTCCAAGAAGGAGATCAAAGAC | CGACGAGGGTCAGCTACCA |
| (ribosomal protein | (SEQ ID No. 57) | (SEQ ID No. 58) |
| S16) | | |

After the completion of the PCR reaction, the mRNA (as represented by the amount of PCR-amplified cDNA detected) for one or more markers is quantified. In certain embodiments, quantification is calculated by comparing the amount of mRNA encoding a disease marker to a reference value. In some embodiments the reference value will be the amount of mRNA found in healthy non-diseased patients. In other embodiments, the reference value is the expression level of a house-keeping gene. In certain such embodiments, beta-actin, or other appropriate housekeeping gene is used as the reference value. Numerous other house-keeping genes that are well known in the art may also be used as a reference value. In other embodiments, a house keeping gene is used as a correction factor, such that the ultimate comparison is the expression level of marker from a diseased patient as compared to the same marker from a non-diseased (control) sample. In several embodiments, the house keeping gene is a tissue specific gene or marker, such as those discussed above. In still other embodiments, the reference value is zero, such that the quantification of the markers is represented by an absolute number. In several embodiments a ratio comparing the expression of one or more markers from a diseased patient to one or more other markers from a non-diseased person is made.

In alternative embodiments, the ability to determine the total efficiency of a given sample by using known amounts of spiked standard RNA results from embodiments being dose-independent and sequence-independent. The use of known amounts of control RNA allows PCR measurements to be converted into the quantity of target mRNAs in the original samples.

In several other embodiments, expression of markers related to diabetic nephropathy is measured before and/or after administration of a drug (or other therapy) to a patient. In certain such embodiments, the expression profiles may be used to predict the efficacy of a drug compound (e.g. in treating diabetic nephropathy) or to monitor side effects of the drug compound (e.g., impact on kidney function). In some embodiments, the drug monitored may have been administered to treat one or more of chronic kidney disease, acute renal failure, diabetic nephropathy, glomerulonephritis, glomerulosclerosis, focal segmental glomerulosclerosis, membranous nephropathy, minimal change disease, atherosclerosis, hypertension, cardiovascular diseases, obesity, hypercholesterolemia, diabetes, collagen diseases, cancer drug, infections, and/or immunosuppressive diseases. In some embodiments, a drug compound will induce the expression of a distinctive mRNA profile. Likewise, in other embodiments, a drug may inhibit expression of one or more markers. In some such embodiments, the efficacy of drug treatment can be monitored by the disappearance (or reduced expression) of markers associated with a particular disease state. In several embodiments, the methods disclosed herein evaluate a change in diet, lifestyle, or other non-traditional (e.g., non-drug) therapy on the function of a diabetic subject's kidneys.

In several embodiments, the analyses described herein are applicable to human patients, while in some embodiments, the methods are applicable to animals (e.g., veterinary diagnoses).

### Implementation Mechanisms

According to some embodiments, the methods described herein can be implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, server computer systems, portable computer systems, handheld devices, networking devices or any other device or combination of devices that incorporate hard-wired and/or program logic to implement the techniques.

Computing device(s) are generally controlled and coordinated by operating system software, such as iOS, Android, Chrome OS, Windows XP, Windows Vista, Windows 7, Windows 8, Windows Server, Windows CE, Unix, Linux, SunOS, Solaris, iOS, Blackberry OS, VxWorks, or other compatible operating systems. In other embodiments, the computing device may be controlled by a proprietary operating system. Conventional operating systems control and schedule computer processes for execution, perform memory management, provide file system, networking, I/O services, and provide a user interface functionality, such as a graphical user interface (“GUI”), among other things.

In some embodiments, the computer system includes a bus or other communication mechanism for communicating information, and a hardware processor, or multiple processors, coupled with the bus for processing information. Hardware processor(s) may be, for example, one or more general purpose microprocessors.

In some embodiments, the computer system may also includes a main memory, such as a random access memory (RAM), cache and/or other dynamic storage devices, coupled to a bus for storing information and instructions to be executed by a processor. Main memory also may be used for storing temporary variables or other intermediate information during execution of instructions to be executed by the processor. Such instructions, when stored in storage media accessible to the processor, render the computer system into a special-purpose machine that is customized to perform the operations specified in the instructions.

In some embodiments, the computer system further includes a read only memory (ROM) or other static storage device coupled to bus for storing static information and instructions for the processor. A storage device, such as a magnetic disk, optical disk, or USB thumb drive (Flash drive), etc., may be provided and coupled to the bus for storing information and instructions.

In some embodiments, the computer system may be coupled via a bus to a display, such as a cathode ray tube (CRT) or LCD display (or touch screen), for displaying information to a computer user. An input device, including alphanumeric and other keys, is coupled to the bus for communicating information and command selections to the processor. Another type of user input device is cursor control, such as a mouse, a trackball, or cursor direction keys for communicating direction information and command selections to the processor and for controlling cursor movement on display. This input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

In some embodiments, the computing system may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage

In some embodiments, a computer system may implement the methods described herein using customized hard-wired logic, one or more ASICs or FPGAs, firmware and/or program logic which in combination with the computer system causes or programs the computer system to be a special-purpose machine. According to one embodiment, the methods herein are performed by the computer system in response to hardware processor(s) executing one or more sequences of one or more instructions contained in main memory. Such instructions may be read into main memory from another storage medium, such as a storage device. Execution of the sequences of instructions contained in main memory causes processor(s) to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, or other types of storage devices. Volatile media includes dynamic memory, such as a main memory. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between nontransitory media. For example, transmission media includes coaxial cables, copper wire and fiber optics, including the wires that comprise a bus. Transmission media can also take the form of acoustic or light waves, such as those generated during radio-wave and infra-red data communications.

Various forms of media may be involved in carrying one or more sequences of one or more instructions to a processor for execution. For example, the instructions may initially be carried on a magnetic disk or solid state drive of a remote computer. The remote computer can load the instructions into its dynamic memory and send the instructions over a telephone line using a modem or other network interface, such as a WAN or LAN interface. A modem local to a computer system can receive the data on the telephone line and use an infra-red transmitter to convert the data to an infra-red signal. An infra-red detector can receive the data carried in the infra-red signal and appropriate circuitry can place the data on a bus. The bus carries the data to the main memory, from which the processor retrieves and executes the instructions. The instructions received by the main memory may retrieve and execute the instructions. The instructions received by the main memory may optionally be stored on a storage device either before or after execution by the processor.

In some embodiments, the computer system may also include a communication interface coupled to a bus. The communication interface may provide a two-way data communication coupling to a network link that is connected to a local network. For example, a communication interface may be an integrated services digital network (ISDN) card, cable modem, satellite modem, or a modem to provide a data communication connection to a corresponding type of telephone line. As another example, a communication interface may be a local area network (LAN) card to provide a data communication connection to a compatible LAN (or WAN component to communicate with a WAN). Wireless links may also be implemented. In any such implementation, a communication interface sends and receives electrical, electromagnetic or optical signals that carry digital data streams representing various types of information.

A network link may typically provide data communication through one or more networks to other data devices. For example, a network link may provide a connection through a local network to a host computer or to data equipment operated by an Internet Service Provider (ISP). The ISP in turn provides data communication services through the world wide packet data communication network now commonly referred to as the “Internet.” The local network and Internet both use electrical, electromagnetic or optical signals that carry digital data streams. The signals through the various networks and the signals on the network link and through a communication interface, which carry the digital data to and from the computer system, are example forms of transmission media.

In some embodiments, the computer system can send messages and receive data, including program code, through the network(s), the network link, and the communication interface. In the Internet example, a server might transmit a requested code for an application program through the Internet, ISP, local network, and communication interface.

The received code may be executed by a processor as it is received, and/or stored in a storage device, or other non-volatile storage for later execution.

### EXAMPLES

Specific embodiments will be described with reference to the following examples which should be regarded in an illustrative rather than a restrictive sense.

### Example 1

### Identification of Biomarkers Associated with Diabetic Nephropathy

For many diabetic patients, early diagnosis of kidney problems followed by appropriate treatment or strict blood glucose control is only way to prevent end-stage kidney disease. As discussed above, however, kidney function tests are relatively limited and often insufficiently sensitive to detect early signs of kidney problems. The invasive nature of kidney precludes its use as routine diagnostic test.

Given ready access to potentially large quantities of patient urine samples, several embodiments of the methods disclosed herein employ urine as a diagnostic sample. Many current diagnostic tests measure solutes excreted in urine, or measure urine production rate, in order to evaluate kidney function, or loss thereof. However, several embodiments of the methods disclosed herein exploit the presence of nucleic acid-containing vesicles present in the urine make a sensitive and specific diagnostic analysis of kidney function based on isolation and amplification of kidney specific markers.

### Methods

### Samples

Urine samples were obtained from healthy donors (n=23) and diabetic nephropathy patients (n=23) at the hospital of University of California San Diego.

Exosomal mRNA Analysis.

Each urine sample was centrifuged at 1,000×g for 15 minutes, and 10 mL of the resulting supernatant was applied (by vacuum) to a 96-well exosome-capture filterplate. The filterplate was then centrifuged at 2,000×g for an additional 5 minutes. In each well, 60 μL of Lysis buffer containing a cocktail of antisense primers were added, and incubated at 55° C. for 10 minutes. The resultant lysate was transferred from the filterplate to an oligo(dT)-immobilized 96-well microplate by centrifugation at 2,000×g for 5 minutes. cDNA was directly synthesized in the same oligo(dT)-immobilized 96-well microplate by adding dNTPs (final concentration of 5 mM), MMLV reverse transcriptase (final concentration of 2.7 U/mL), and RNasin (final concentration of 0.13 U/mL) (Invitrogen, Carlsbad, Calif.) and incubation at 37° C. for 2 hours. cDNA was subsequently used in real time SYBR green PCR using iTaqSYBR master mix (BioRad, Hercules, Calif.) by established methods (see e.g., Mitsuhashi M, J Immunol Methods. 363:95-100, 2010, which is incorporated in its entirety by reference herein). PCR conditions were 50 cycles of annealing at 65° C. for 1 minute followed by denaturization at 95° C. for 30 seconds using a PRISM 7900 (Applied Biosystems (ABI), Foster City, Calif.). The results were expressed as the cycle threshold (Ct) using the analytical software (SDS, ABI). Ct=32 was considered as the baseline.

Targeting mRNAs

A total of 23 mRNAs were quantified, and included: 2 control genes (β-actin (ACTB) and β2 microglobulin (B2M)), glomerulus-specific (podocine (PDCN)), proximal tubules-specific (uromodulin (UMOD), albumin (ALB), and Na/K/Cl transporter (SLC12A1)), distal tubules-specific (aquaporin 9 (AQP9)), as well as kidney-diabetes related miscellaneous mRNAs (peroxisome proliferator-activated receptor gamma, coactivator 1α and β (PPARGC1A and B), nuclear respiratory factor 1 and 2 (NRF1 and 2), estrogen-related receptor α (ESRRA), annexin A5 (ANXA5), protein kinase, AMP-activated, α1 and α2 catalytic subunit (PRKAA1 and 2), uncoupling protein 1 and 2 (UCP1 and 2), low density lipoprotein receptor-related protein 2 (LRP2), CD24, secreted phosphoprotein 1 (SPP1), α2-HS-glycoprotein (AHSG), and SMAD family member 1 (SMAD1)).

### Results

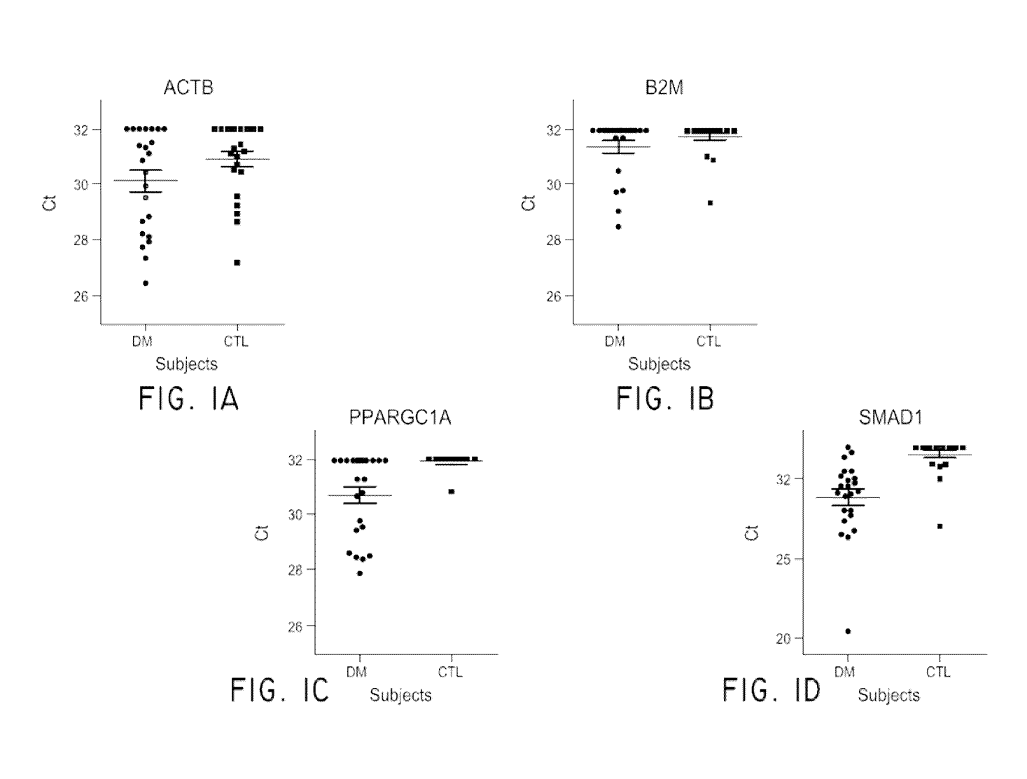

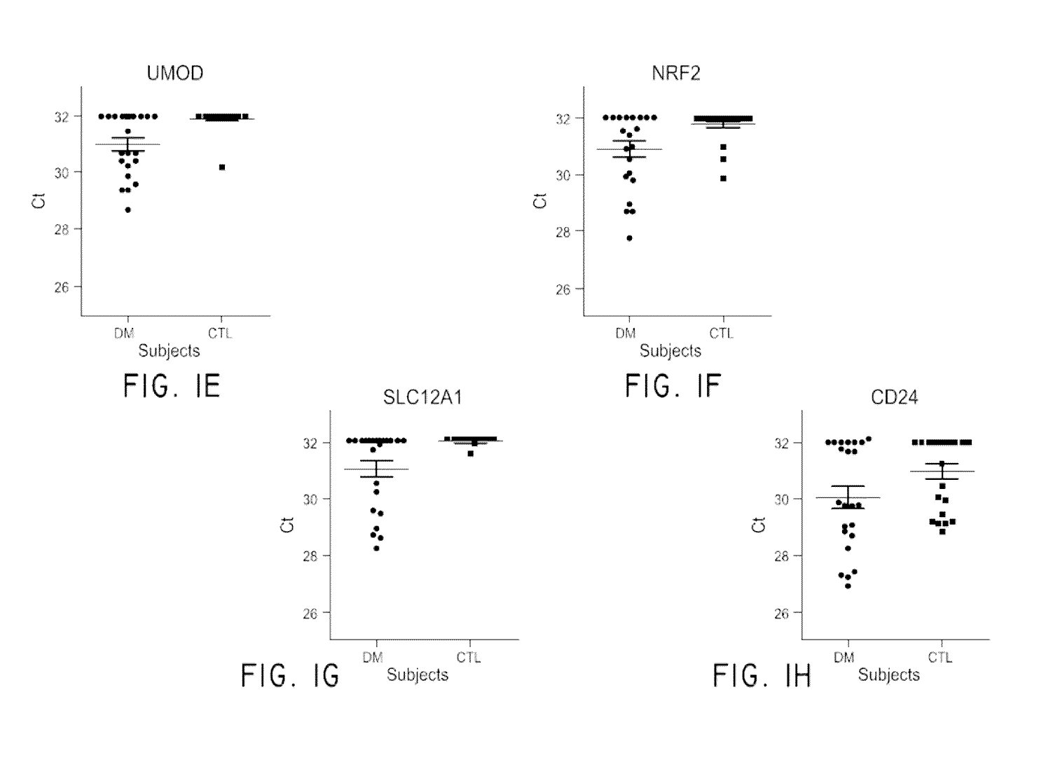

Expression levels of various exosomal mRNA from either diabetic (DM) or normal (CTL) patients were compared (FIGS. 1A-1H). As shown in FIG. 1A/1B, expression of the control genes (β-actin, ACTB) and B2M did not differ based on presence or absence of diabetes. In contrast, significant increases in expression of PPARGC1A, SMAD1, UMOD, NRF2, SLC12A1 and CD24 were detected in DM patients. These data indicate that these mRNAs (as well as others that are increased in response to diabetes-induced, or other type, of kidney damage) are correlated with the presence of diabetes. Their upregulation indicate their possible utility as biomarkers associated with the disease and its related loss of kidney function.

In order to characterize the severity of the diabetic condition in each DM patient, exosome mRNA expression data was correlated with results of HbA1c testing. The HbA1c test evaluates the blood glucose levels in a diabetic patient over time. Clinicians generally view an HbA1c of 5.6% or less is normal. Ranges of 5.7% to 6.4% are associated with pre-diabetes, while levels of 6.5% or higher leads to a diagnosis of diabetes. Above 6.5%, increased HbA1c levels are correlated with increasingly severe dysregulation of blood glucose, and thus increased risk for diabetic nephropathy.

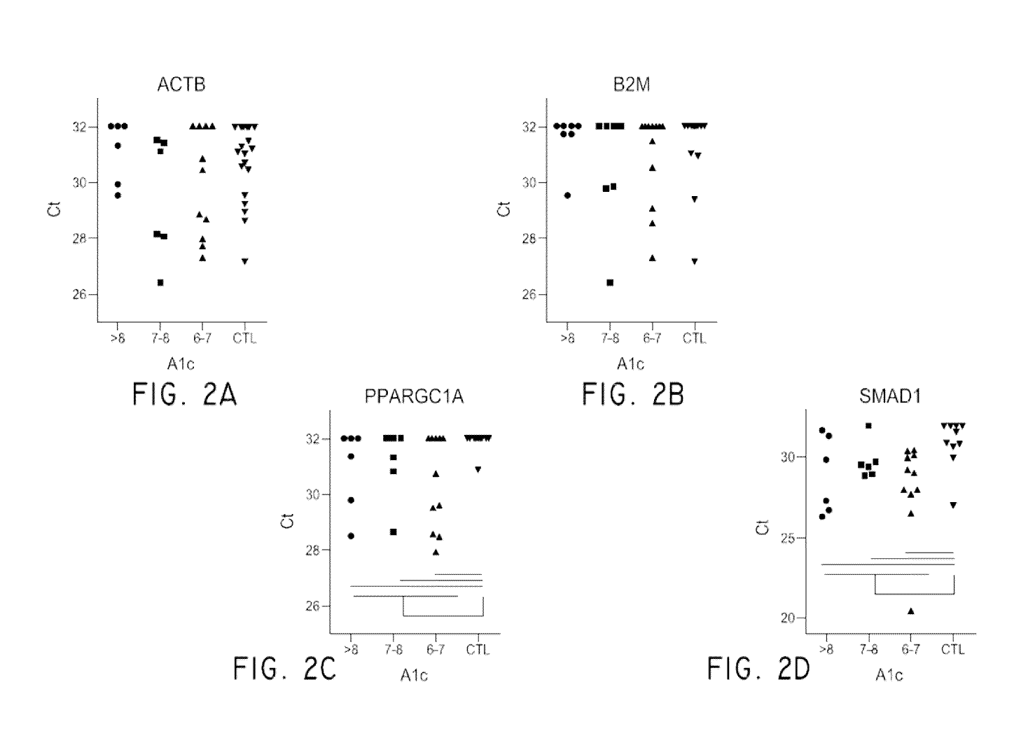

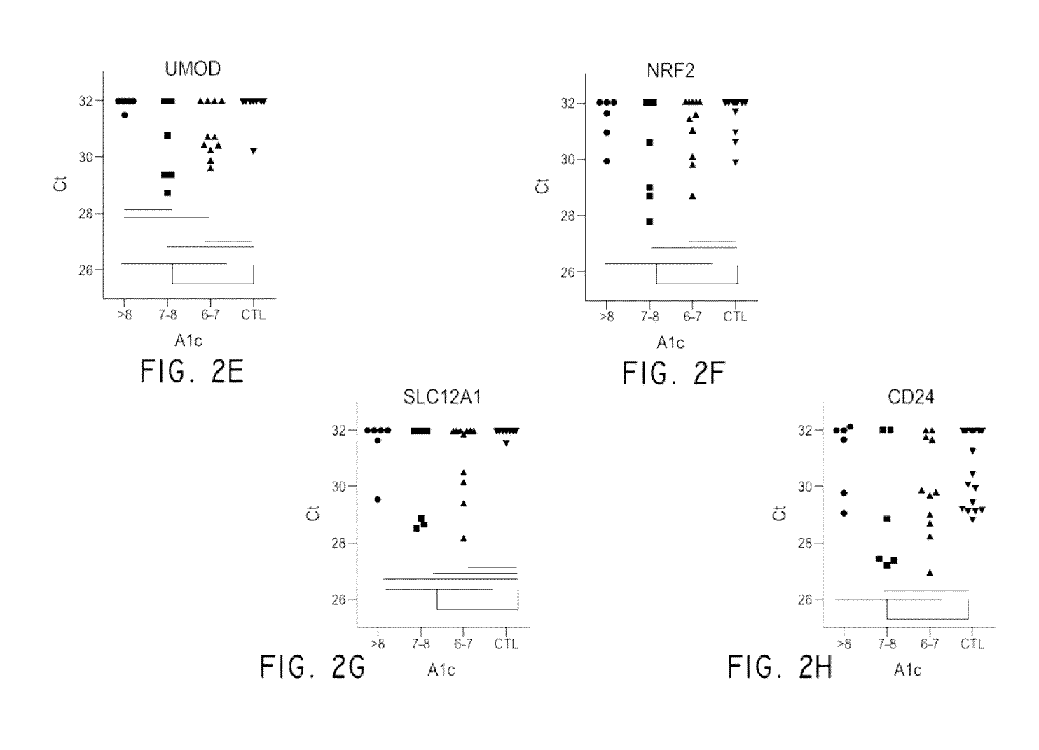

As shown in FIGS. 2A-2H, even those DM patients with only a slight increase of HbA1c (6-7%) demonstrated significant increased expression (versus healthy controls) of 5 genes (PPARGC1A, SMAD1, UMOD, NRF2, SLC12A1, not CD24). Given the modest increase in HbA1c levels, these data suggest that these mRNAs are sensitive biomarkers of DM.

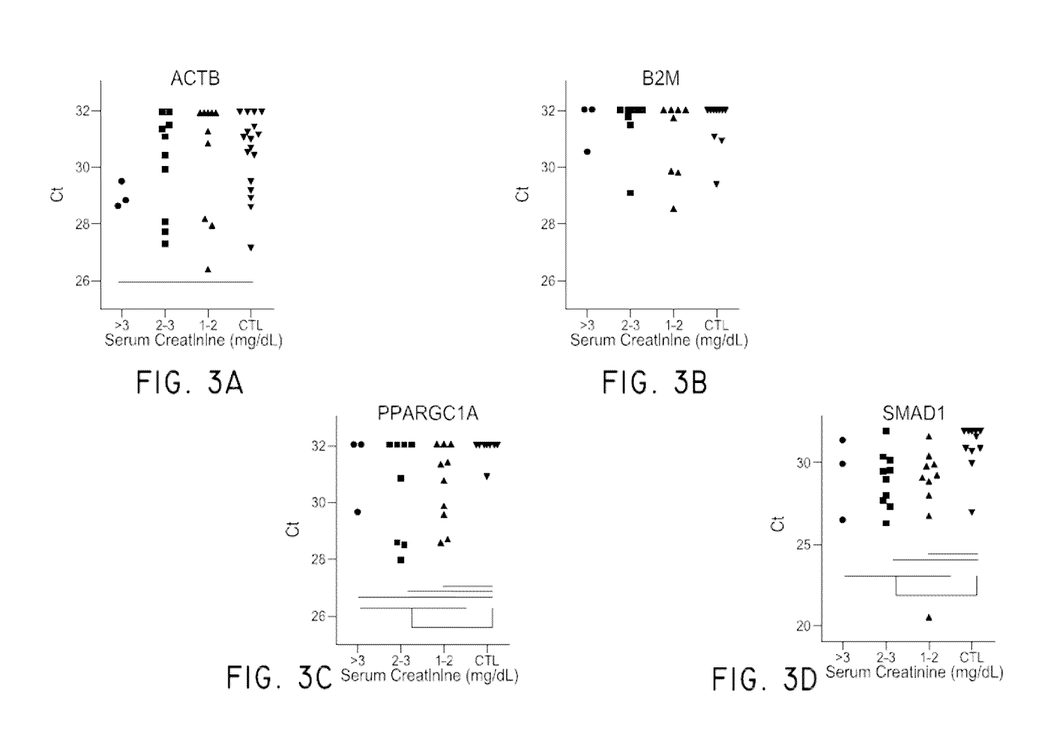

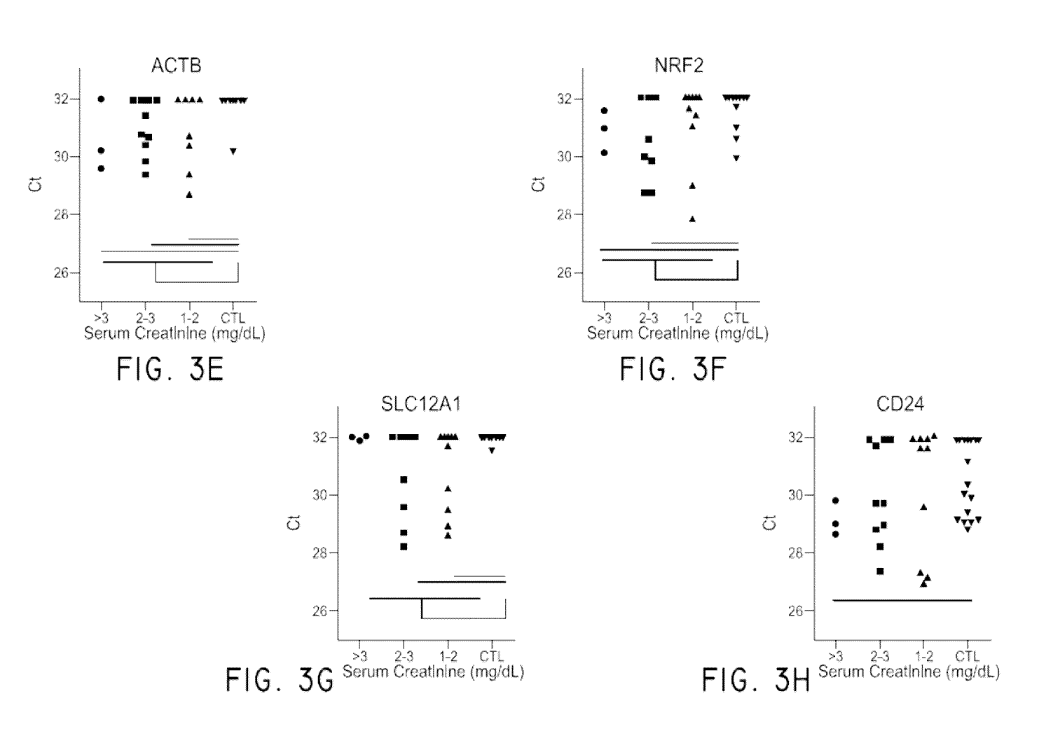

To further evaluate candidate biomarkers of kidney damage, urine exosome mRNAs were then compared with the levels of serum creatinine. Creatine phosphate is metabolized by the muscles to produce energy and creatinine is produced as a waste product. Creatinine is carried by the blood to the kidneys where it is excreted in the urine. Generally, since creatinine production is linked to muscle mass, which varies little from day to day, so creatinine level should remain relatively constant if kidneys are functioning properly. Increased creatinine levels are indicative of reduced filtration, which is a hallmark of diabetic-induced damage to the nephrons. As shown in FIGS. 3A-3H, DM patients with only a slight increase in serum creatinine (1-2 mg/dL) still showed significant differences in gene expression (against healthy controls) in 4 genes (PPARGC1A, SMAD1, UMOD, SLC12A1). As with the HbA1c data, these results suggest that mRNAs are sensitive biomarkers of kidney damage. Thus, these markers, or others that are elevated in the early stages of kidney damage are used in several embodiments to identify kidney damage at its earliest stages (before other analytical methods would detect severe damage and prior to detectable symptoms).

### Discussion

The data presented above demonstrate that certain mRNA markers can be isolated from patient urine samples, processed, quantified, and correlated to diabetic nephropathy. These data also indicate that certain markers correlated with established diagnostic markers used to identify patients suffering from diabetic nephropathy. As shown in FIG. 1, clear differences in expression levels could be in certain markers when normal subjects were evaluated in comparison to subjects with diabetes. FIGS. 2 and 3 demonstrate that several markers are indicative of the severity of the damaged to the kidney due to diabetes. As shown, the mRNA markers are correlated with traditional diagnostic endpoints. Advantageously, however, the methods disclosed herein are non-invasive (whereas traditional tests require blood draws) can easily and routinely be repeated, and are highly sensitive. This sensitivity, as discussed above, is particularly advantageous as it allows the early detection of diabetic nephropathy, in many cases prior to the ability of a patient or doctor to identify symptoms. As such, in several embodiments, the claimed methods allow preventative action to take place (e.g., lifestyle change, more robust therapy to control the diabetes etc.) and thus prevent disease progression, or at least reduce the severity of the progression. In addition, the high degree of correlation with currently used clinical markers, allows the methods disclosed herein to be used to identify additional genetic markers of diabetic nephropathy, including those that are indicative of the severity of the disease.

### Example 2

### Identification of Biomarkers Associated with Diabetic Nephropathy

Diabetic nephropathy, or diabetic kidney disease (DKD) takes many years to develop. The earliest sign of kidney disease is indicated by the presence of small amounts of albumin in the urine, called microalbuminuria. Not all individuals with microalbuminuria, however, progress to end stage renal disease. Diagnostic biomarkers with improved sensitivity are necessary. This study was conducted in order to evaluate urine exosome mRNA as potential biomarkers in screening Type II diabetes patients for kidney disease. Exosomes and microvesicles (EMVs) from 10 mL urine (n=2 control, n=3 DKD) were captured and collected by a filter device called ExoComplete Isolation Tube. The EMV mRNAs were released by a lysis buffer and hybridized to a T7 promoter-linked oligo(dT) coated plate. The RNA was captured, amplified by in vitro transcription in solid phase, and then used as starting material for next generation RNA sequencing library preparation. Using CyberT software for data analysis, the most highly significant differentially expressed mRNA were those encoding for cytosolic ribosomes and mitochondrial components involved in translational elongation and oxidative phosphorylation, respectively. Validation by qPCR using 22 control and 18 DKD patient urine samples confirmed six potential mRNA biomarkers. Several of these biomarkers have been implicated to play a role in mediating oxidative stress. This study suggests that one or more of these markers may have a role in the progression of end stage renal disease.

### Methods

### Samples

Urine samples were obtained from healthy donors (n=2) and patients with stages >3 diabetic kidney disease (n=3) at the University of California, San Diego Medical Center.

Exosomal mRNA Analysis.

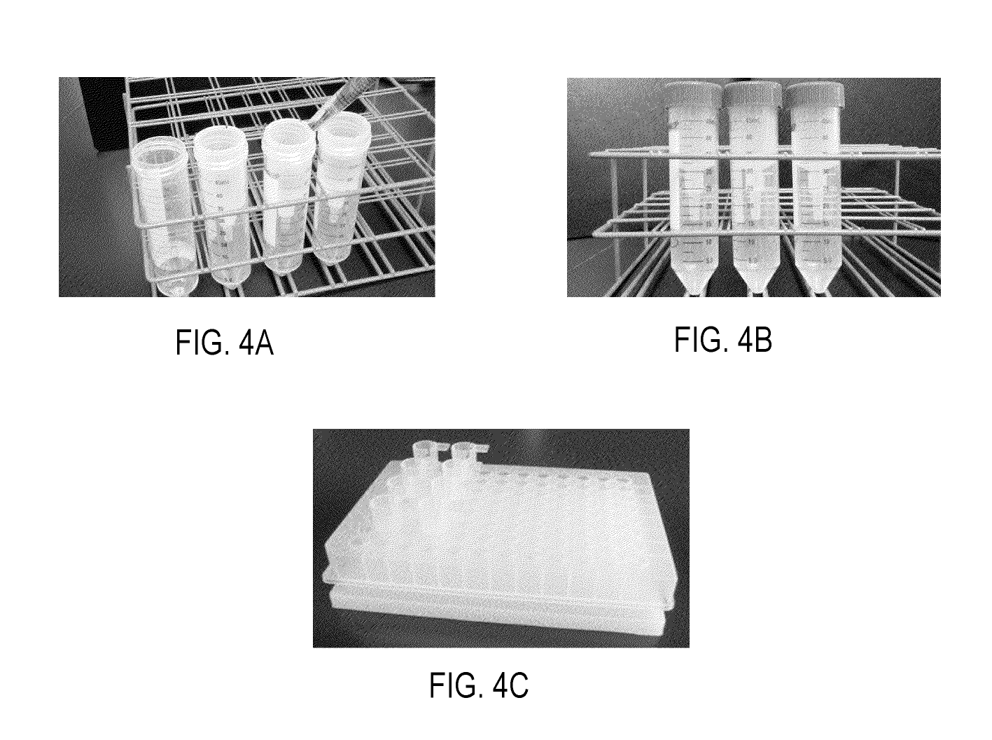

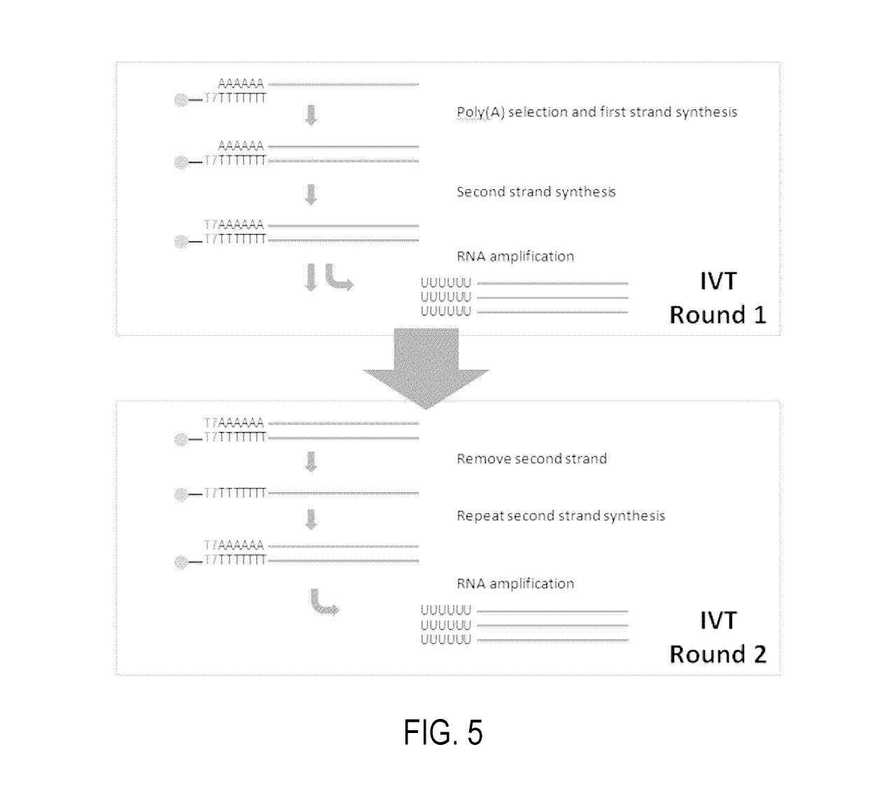

A low speed spin at 800×g for 15 min removed cells and debris from each sample. A ¼ volume of 25×PBS and 10 mL supernatant was applied to the Exosome Isolation Tube (Hitachi Chemical Diagnostics, Inc.) and then centrifuged (FIGS. 4A and B). The captured exosomes were lysed on the filter tip, and the resultant lysates were transferred by centrifugation to a T7 promoter oligo(dT)-immobilized microplate for mRNA hybridization (FIG. 4C). The hybridized mRNA was amplified by in vitro transcription (Megascript, Life Technologies) (FIG. 5) directly on the plate, and then used as starting material for TruSeq library preparation (IIlumina). A single read 50 was run on an Illumina HiSeq 2500 instrument. Data analysis was performed using CyberT software (Baldi P and Long AD, Bioinformatics, 17, 6:509-519, 2001).

### Results

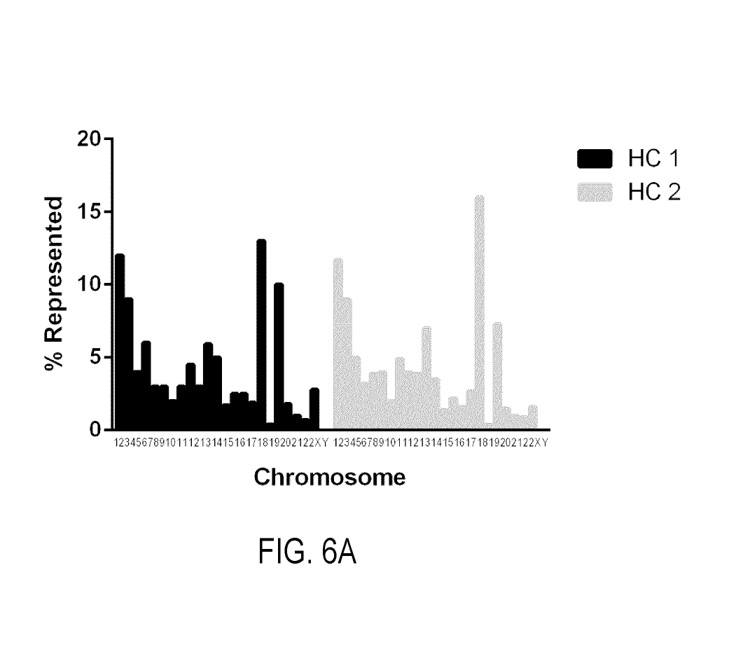

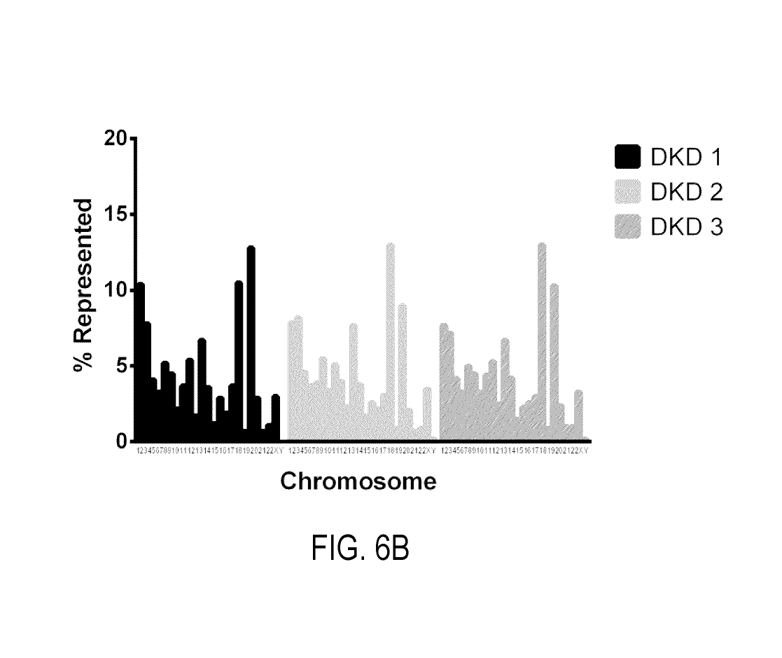

Using CASAVA 1.8.2, the sequencing run was aligned to the UCSC reference genome assembly, hg19. The Illumina TruSeq-prepared libraries had between 30-54 million reads and >9,000 genes. The genes were mapped back to the human chromosomes and represented as the % reads aligning to each chromosome (FIG. 6A-B). All chromosomes of the human genome are represented.

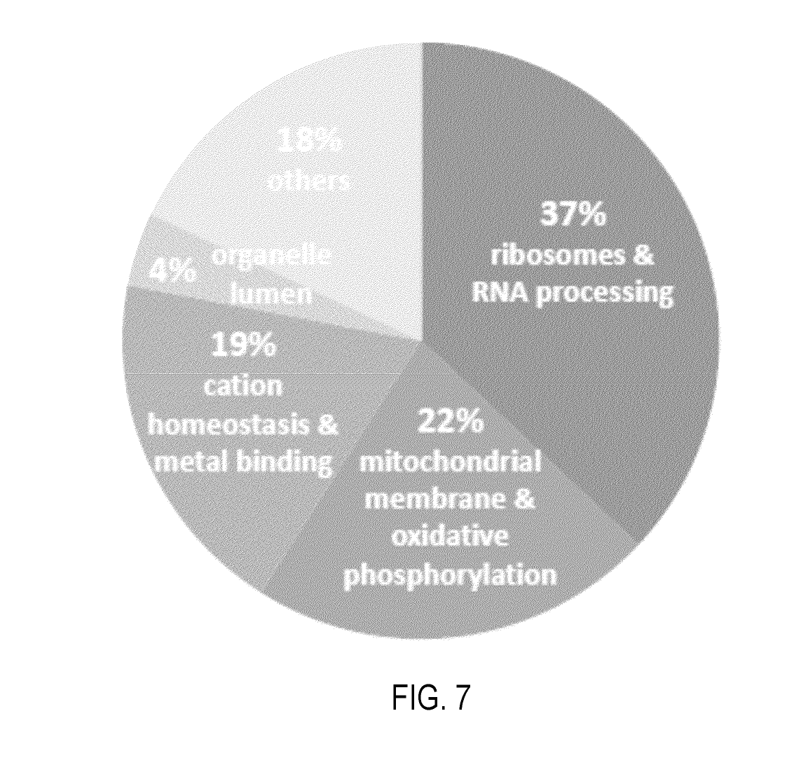

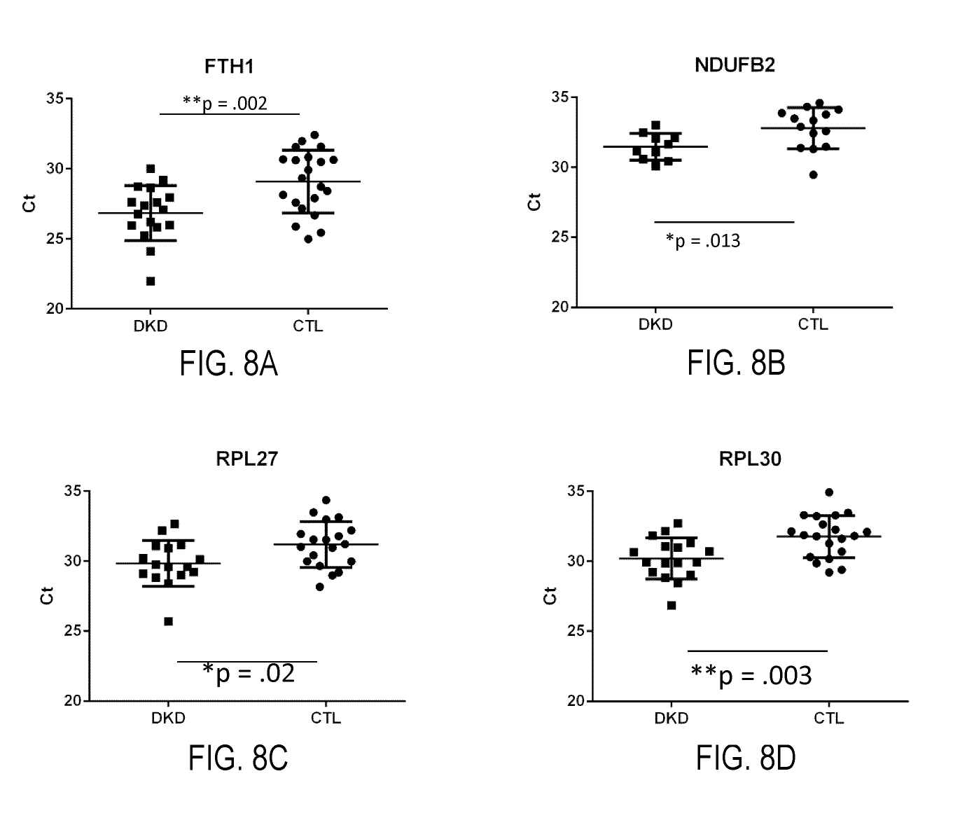

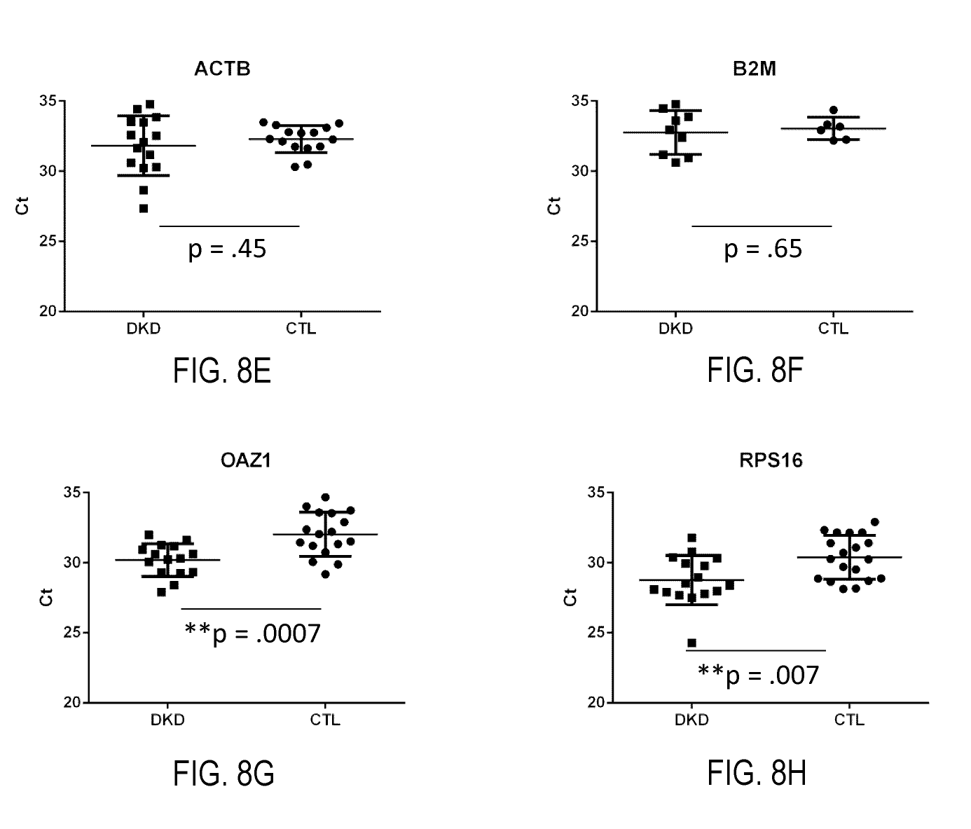

There were 27 genes that were differentially up-regulated in DKD samples compared to controls (12 genes with p<0.01, 15 genes with p<0.05). Using DAVID 6.7 web-based program (Nature Protocols 2009; 4(1):44 & Nucl Acids Res. 2009; 37(1):1), the 27 genes were analyzed under high stringency conditions for functional-related gene groups (FIG. 7). Six of the 27 genes were validated by RT-qPCR using 40 clinical urine samples (22 HC, 18 DKD) (FIGS. 8A-H) and determined to be statistically significant using a GraphPad software unpaired t-test (*p<0.05, **p<0.005).

### Discussion

Exosome mRNA from urine obtained from healthy and DKD patients were prepared for next generation sequencing. The percentage of gene expression based on RPKM values for each chromosome was similar in each of the samples. Differential gene expression analysis identified 27 mRNA that were up-regulated in DKD patients. Functional annotation clustered 37% in ribosomes and RNA processing, 22% as mitochondrial components and oxidative phosphorylation, and 19% in cation homeostasis and metal binding. RT-qPCR on 40 clinical urine samples validated 6 of the 27 possible biomarkers. Housekeeping genes such as ACTB and B2M remained unchanged between healthy controls and DKD samples. This study demonstrates several advantages of using exosome isolation and analysis to identify urinary exosome mRNA that may be used as DKD biomarkers.

### Example 3

### Normalization of Biomarkers Associated with Diabetic Kidney Disease

### Methods

### Samples

Urine samples collected from various subjects were immediately brought to the designated laboratory and stored in a −80° C. freezer without any centrifugation.

Exosomal mRNA Analysis.

Human urine samples stored frozen in 50 cc conical tubes were thawed in a 37° C. water bath before processing. Urine samples were centrifuged at 800×g for 15 min to remove cells and larger particles. The supernatants were transferred to a separate tube and mixed with a ¼ volume of 25×PBS, pH 7.4 (Thermo Scientific, Waltham, Mass.). This mixture was applied to EV collection tubes (Hitachi Chemical Diagnostics, Inc., Mountain View, Calif.) and centrifuged at 2,000×g for 10 min. The EV-containing filter tip was removed from the tube and placed over an oligo(dT)-immobilized microplate via a filter tip holder. One hundred μL working lysis buffer containing 10 nM antisense primer (Integrated DNA Technologies, Coralville, Iowa) of each target mRNA, was applied to each filter-tip and incubated at 37° C. for 10 min. Lysates were transferred to the microplate by centrifugation and incubated at 4° C. overnight for mRNA hybridization. After a series of six wash steps, gene-specific reverse transcription reactions on the hybridized mRNA were performed by adding 100 μL cDNA reaction mixture containing 1× reverse transcriptase buffer, 1.25 mM dNTPs, 2.7 U/μL MMLV reverse transcriptase and 0.13 U/μL RNasin (Promega, Madison, Wis.) to each well and incubating at 37° C. for 2 hours. Two μL of cDNA was used for real-time PCR analysis in a 5 μL reaction volume containing 1× SsoAdvanced SYBR Green Supermix (Bio-Rad, Hercules, Calif.) and 500 nM primer pairs (synthesized by Integrated DNA Technologies, Coralville, Iowa). The primer sequences are available in Table 1. Real-time PCR was performed on a ViiA7 (Life Technologies, Carlsbad, Calif.) instrument using the following profile: initial denaturation at 95° C. for 10 min, 40 cycles of 95° C. for 30 sec and 65° C. for 1 min, melting curve analysis. Threshold cycle was set by instrument software program. Comparison data between severe DKD subjects and healthy controls are shown in Table 2.

Real-time PCR data was normalized by ACTB, urinary creatinine or the mean of a trio of ribosomal genes (RPS16, RPL27, RPL30). The normalization with creatinine was performed by subtraction of log 2 (creatinine value in mL/min) from target gene Ct values. The normalization with the ACTB and ribosomal genes was performed by subtracting the Ct value of ACTB or ribosomal gene from the Ct value of the target gene. Table 2 shows mean normalized PCR data for the severe DKD group and for the healthy non-obese group. Table 2 also shows statistical significance (indicated by p-value) of normalized mRNA expression between the two groups for each target gene.

TABLE 2
Normalized mRNA expression in severe DKD subjects (n = 16) and healthy controls
(n = 20).

| | Ribo Trio Normalized | | | Log-Creatinine | | | | | |
|---|---|---|---|---|---|---|---|---|---|
| | | Healthy | | Normalized | | | ACTB Normalized | | |
| | Severe | non- | | Severe | Healthy | | Severe | Healthy | |
| | DKD | obese | | DKD | non-obese | | DKD | non-obese | |
| | Group | Group | | Group | Group | | Group | Group | |
| mRNA | Mean | Mean | p-value | Mean | Mean | p-value | Mean | Mean | p-value |
| ACTB | 0.74 | 0.84 | 0.76 | 20.1 | 21.1 | 0.14 | 0 | 0 | N/A |
| B2M | 1.88 | 2.08 | 0.67 | 21.3 | 22.3 | 0.12 | 1.14 | 1.24 | 0.68 |
| CD24 | 1.38 | 1.56 | 0.39 | 20.8 | 21.8 | 0.12 | 0.65 | 0.72 | 0.81 |
| FTH1 | −2.98 | −2.99 | 0.98 | 16.4 | 17.2 | 0.15 | −3.72 | −3.82 | 0.66 |
| NDUFB2 | 1.33 | 1.77 | 0.004 | 20.7 | 22 | 0.06 | 0.6 | 0.93 | 0.37 |
| NRF2 | 3.35 | 3.56 | 0.55 | 22.7 | 23.8 | 0.13 | 2.62 | 2.72 | 0.74 |
| OAZ1 | 0.59 | 0.78 | 0.16 | 20 | 21 | 0.09 | −0.14 | −0.06 | 0.75 |
| PPARGC1 | 2.92 | 3.25 | 0.34 | 22.3 | 23.5 | 0.09 | 2.19 | 2.41 | 0.59 |
| RPL27 | −0.41 | −0.5 | 0.1 | 19 | 19.7 | 0.25 | −1.14 | −1.34 | 0.58 |
| RPL30 | 0.96 | 1 | 0.58 | 20.4 | 21.2 | 0.17 | 0.23 | 0.16 | 0.84 |
| RPS16 | −0.55 | −0.5 | 0.24 | 18.8 | 19.7 | 0.16 | −1.29 | −1.34 | 0.89 |
| SLC12A1 | 2.06 | 3.41 | 0.003 | 21.5 | 23.6 | 0.009 | 1.32 | 2.57 | 0.02 |
| SMAD1 | 5.58 | 5.8 | 0.53 | 25 | 26 | 0.13 | 4.85 | 4.96 | 0.78 |
| UMOD | 2.43 | 4.37 | <0.0001 | 21.8 | 24.6 | 0.001 | 1.7 | 3.53 | 0.001 |

### Statistical Analysis

For predictive model development, real-time PCR data was analyzed using random forest and sparse logistic regression methodology. A detailed description of random forest regression is provided in Frederick Livingston, Implementation of Breiman's Random Forest Machine Learning Algorithm, ECE591Q Machine Learning Journal Paper, Fall 2005, which is incorporated herein by reference in its entirety. Sparse logistic regression is described in Friedman, J., Hastie, T. and Tibshirani, R. (2008) Regularization Paths for Generalized Linear Models via Coordinate Descent, http://www.stanford.edu/˜hastie/Papers/glmnet.pdf, Journal of Statistical Software, Vol. 33(1), 1-22 Feb. 2010, which is incorporated herein by reference in its entirety. Sparse logistic regression is also described in James, G., Witten, D., Hastie, T., Tibshirani, R. (2013) An Introduction to Statistical Learning with Applications in R. Springer-Verlag New York, which is incorporated herein by reference in its entirety

Table 3 shows the characteristics of the subject groups used to train and test classifiers for the predictive model. Estimated glomerular filtration rate (eGFR) is in mL/min. Urinary albumin/creatinine ratio (ACR) is in mg albumin/g creatinine.

TABLE 3
Subject groups used to train and test predictive models.

| Category | Group | N | Subjects |
|---|---|---|---|
| Positive control | i | 20 | DKD with eGFR > 54, ACR > 24 |
| Positive control | ii | 16 | DKD with eGFR 12-54, ACR > 24 |
| Positive control | iii | 12 | Non-diabetic CKD with eGFR 12-54, |
| | | | ACR > 24 |
| Negative control | iv | 20 | Obese non-DM with eGFR > 54, A1c < |
| | | | 6.5%, ACR <= 36 |
| Negative control | v | 20 | Non-obese, non-DM adult volunteers, |
| | | | eGFR > 54, A1c < 6.5%, ACR <= 36 |
| Target | vi | 97 | Type II DM with eGFR > 54, A1c <= |
| | | | 8%, ACR <= 36 |
| Target | vii | 77 | Type II DM with eGFR > 54, A1c > |
| | | | 8%, ACR <= 36 |

In Tables 4A-B, random forest and sparse logistic regression analysis was used to train classifiers from subjects with severe diabetic kidney disease (DKD) and healthy controls (groups ii and v, Table 3). The model was then applied to mild DKD and obese healthy controls (groups i and iv, Table 3). Results are shown as area under the curve (AUC) for training and test set, sensitivity and specificity. The reported sensitivity and specificity were estimated with a threshold of 0.5.

The random forest analysis uses all 14 genes listed in Table 2. The sparse logistic regression analysis uses only selected genes. For the sparse logistic analysis, the genes are selected using an elastic-net regularization technique. This technique adds a penalty function for including too many genes in the model. In other words, the best model fits the data well and is parsimonious, in terms of having the smallest number of genes possible. One piece of the penalty function (the L1 norm) is the number of genes used in the model; the second piece of the penalty function (the L2 norm) is sum of the squared coefficients of the genes in the model. The use of the two pieces in the elastic-net regularization has been shown to have good properties and avoid some of the computational problems encountered with just using the L1 norm. For the ACTB normalized data, the sparse logistic regression uses the following genes: B2M, CD24, FTH1, NRF1, NRF2, OAZ1, PPARGC1A, PPARGC1B, RPL27, SMAD1, and UMOD. For the ribosomal trio normalized data, the sparse logistic regression uses the following genes: CD24, FTH1, NDUFB2, NRF1, NRF2, OAZ1, PPARGC1A, PPARGC1B, RPL27, RPL30, SMAD1, and UMOD. For the creatinine normalized data, the sparse logistic regression uses UMOD.

TABLE 4A
Random forest method.

| | Training | Test set | Test set | Test set |
|---|---|---|---|---|
| Data | set AUC | AUC | sensitivity | specificity |
| ACTB normalized | 0.63 | 0.72 | 65% | 75% |
| Ribosomal trio normalized | 0.82 | 0.72 | 65% | 75% |
| Creatinine normalized | 0.56 | 0.68 | 60% | 60% |

TABLE 4B
Sparse logistic regression method.

| | Training | Test set | Test set | Test set |
|---|---|---|---|---|
| Data | set AUC | AUC | sensitivity | specificity |
| ACTB normalized | 0.92 | 0.74 | 60% | 75% |
| Ribosomal trio normalized | 0.92 | 0.77 | 65% | 65% |
| Creatinine normalized | 0.86 | 0.73 | 75% | 75% |

In Tables 5A-B, random forest and sparse logistic regression analysis was used to train classifiers from subjects with mild and severe DKD (groups i/ii, Table 3) and obese and non-obese healthy controls (groups iv/v, Table 3). The model was then applied to mild DKD and obese healthy controls (groups i and iv, Table 3). Results are shown as AUC for training and test set, sensitivity and specificity.

TABLE 5A
Random forest method - cross validated estimates.

| Data | AUC | sensitivity | specificity |
|---|---|---|---|
| ACTB normalized | 0.68 | 61% | 73% |
| Ribosomal trio normalized | 0.73 | 72% | 70% |
| Creatinine normalized | 0.62 | 61% | 55% |

TABLE 5B
Sparse logistic regression method - cross validated estimates.

| Data | AUC | sensitivity | specificity |
|---|---|---|---|
| ACTB normalized | 0.80 | 69% | 79% |
| Ribosomal trio normalized | 0.80 | 66% | 80% |
| Creatinine normalized | 0.78 | 68% | 76% |

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a blood test” include “instructing the administration of a blood test.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 3 mm” includes “3 mm.”

What is claimed is:

1. A method for identifying a subject likely to develop or currently affected by diabetic nephropathy, comprising:
obtaining a sample of urine from a subject,
wherein said sample comprises vesicles that are associated with RNA;
isolating the vesicles from said sample;
lysing said vesicles to release said vesicle-associated RNA, wherein said vesicle-associated RNA comprises an RNA associated with diabetes-induced damage to the nephron,
wherein said RNA associated with diabetes-induced damage to the nephron is selected from the group consisting of PPARGC1A, SMAD1, UMOD, NRF2, SLC12A1 and CD24;
quantifying said RNA associated with diabetes-induced damage to the nephron by a method comprising:
(i) contacting RNA from said sample with a reverse transcriptase to generate complementary DNA (cDNA), and
(ii) contacting said cDNA with sense and antisense primers that are specific for one of PPARGC1A, SMAD1, UMOD, NRF2, SLC12A1, and CD24 and a DNA polymerase to generate amplified DNA;
comparing the amount of quantified RNA associated with diabetes-induced damage to the nephron from said sample to the quantity of a corresponding RNA from individuals having normal kidney function; and
identifying a subject as likely to develop or currently affected by diabetic nephropathy when there is a difference in the quantity of said RNA associated with diabetes-induced damage to the nephron between said subject and said the quantity of said RNA in individuals with normal kidney function.

2. The method of claim 1, wherein the difference in the quantity of RNA associated with diabetes-induced damage to the nephron is correlated with one or more non-molecular indicators of diabetic nephropathy.

3. The method of claim 1, wherein said RNA associated with diabetes-induced damage to the nephron is selected from the group consisting of PPARGC1A, SMAD1, UMOD, and SLC12A1.

4. The method of claim 1, wherein isolating the vesicles from said sample comprises filtering the urine.

5. The method of claim 4, wherein said filtration traps said vesicles on the filter.

6. The method of claim 5, wherein said lysing is performed while said vesicles are trapped on said filter.

7. The method of claim 1, further comprising centrifuging said sample to remove cellular debris.

8. The method of claim 7, wherein said centrifugation is performed prior to isolating the vesicles.

9. The method of claim 7, wherein concentrating the vesicles further comprises filtering the supernatant of said centrifuged urine.

10. The method of claim 1, wherein said diabetic nephropathy is due to Type I or Type II diabetes.

11. The method of claim 10, wherein said Type I or Type II diabetes has not yet been diagnosed.

12. The method of claim 1, wherein said RNA associated with diabetes-induced damage to the nephron comprises poly(A)+ RNA.

13. A method according to claim 1, wherein the method is used to screen a plurality of subjects to determine their likelihood of developing diabetic nephropathy and/or to detect early stage diabetic nephropathy.

14. A method for identifying a subject affected by diabetic nephropathy, comprising:
obtaining a sample of urine from a subject, wherein said sample comprises vesicles that are associated with RNA;
isolating the vesicles from said sample;
lysing said vesicles to release said vesicle-associated RNA, wherein said vesicle-associated RNA comprises a target RNA,
wherein said target RNA is selected from the group consisting of B2M, FTH1, PPARGC1A, PPARGC1B, SMAD1, UMOD, NRF1, NRF2, SLC12A1, OAZ1, RPL27, RPL30, NDUFB2, CD24, PPARGC1A, SMAD1, UMOD, NRF2, SLC12A1 and CD24;
quantifying said target RNA such as by using a method selected from the group consisting of reverse-transcription polymerase chain reaction (RT-PCR), real-time RT-PCR, northern blotting, fluorescence activated cell sorting, ELISA, mass spectrometry, and western blotting; and
comparing the amount of said target RNA from said subject to the quantity of a corresponding RNA from individuals having normal kidney function, wherein a difference in the quantity of said target RNA between said subject and said individuals indicates the subject is affected by diabetic nephropathy.

15. A method for determining the progression of diabetic nephropathy in a patient comprising:
obtaining a first sample of urine from a patient at a first time and a second sample of urine from said patient at a second time that is after said first time; wherein said samples comprise vesicles that are associated with RNA;
isolating the vesicles from said samples;
lysing said vesicles to release said vesicle-associated RNA;
wherein said vesicle-associated RNA comprises at least one target RNA and at least one RNA that does not change in response to diabetes-induced damage to the nephron,
wherein said at least one target RNA is selected from the group consisting of B2M, FTH1, PPARGC1A, PPARGC1B, SMAD1, UMOD, NRF1, NRF2, SLC12A1, OAZ1, RPL27, RPL30, NDUFB2, CD24, and combinations thereof;
quantifying said at least one RNA one target RNA said at least one RNA that does not change in response to diabetes-induced damage to the nephron; and
determining a ratio between the amounts of said at least one target RNA and said at least one RNA that does not change in response to diabetes-induced damage to the nephron
identifying progression in said patient's diabetic nephropathy when the ratio of said at least one target RNA to said at least one RNA that does not change in response to diabetes-induced damage to the nephron is increased in said second sample as compared to said first sample.

16. A nucleic-acid based method for detection of early stage diabetic nephropathy, comprising:
obtaining a sample of urine from a subject, wherein said sample comprises vesicles that are associated with RNA;
isolating the vesicles from said sample;
lysing said vesicles to release said vesicle-associated RNA, wherein said vesicle-associated RNA comprises a target RNA,
wherein said target RNA is selected from the group consisting of B2M, FTH1, PPARGC1A, PPARGC1B, SMAD1, UMOD, NRF1, NRF2, SLC12A1, OAZ1, RPL27, RPL30, NDUFB2, and CD24;
quantifying said target RNA such as by using a method selected from the group consisting of reverse-transcription polymerase chain reaction (RT-PCR), real-time RT-PCR, northern blotting, fluorescence activated cell sorting, ELISA, mass spectrometry, and western blotting; and
comparing the amount of said target RNA from said subject to the quantity of a corresponding RNA from individuals having normal kidney function, wherein a difference in the quantity of said target RNA between said subject and said individuals indicates early stage diabetic nephropathy, thereby detecting early stage diabetic nephropathy.

17. The method of claim 16, wherein said detection can be achieved prior to detection by non-nucleic acid detection methods.
